(19) United States
(12) Patent Application Publication
(10) Pub. No.: US 2025/0135159 A1
(43) Pub. Date: May 1, 2025

(54) DYNAMICALLY RIGIDIZING COMPOSITE MEDICAL STRUCTURES

(71) Applicant: Neptune Medical Inc., Burlingame, CA (US)

(72) Inventors: Garrett J. GOMES, Pleasant Hill, CA (US); Alexander Q. TILSON, Burlingame, CA (US); Jeff G. HAYDON, San Mateo, CA (US); Jori J. TYTUS, San Francisco, CA (US); Maya R. EISLER, San Francisco, CA (US); Francisco G. LOPEZ, San Mateo, CA (US); Spencer J. WITTE, San Francisco, CA (US)

(21) Appl. No.: 18/837,186

(22) PCT Filed: Feb. 8, 2023
(86) PCT No.: PCT/US2023/062206

§ 371 Date: Aug. 8, 2024

Related U.S. Application Data
(60) Provisional application No. 63/308,044, filed on Feb. 8, 2022.

(51) Int. Cl. A61M 25/01; A61B 1/005; A61M 25/00

(52) U.S. Cl. CPC A61M 25/0155; A61B 1/0055; A61M 25/005

(57) ABSTRACT

A rigidizing device includes an elongate flexible tube, a rigidizing layer positioned over the elongate flexible tube, an outer layer over the flexible tube and the rigidizing layer, and an inlet between the elongate flexible tube and the outer layer and configured to attach to a source of vacuum or pressure. The rigidizing device is configured to have a rigid configuration when vacuum or pressure is applied through the inlet and a flexible configuration when vacuum or pressure is not applied through the inlet.

### CLAIM OF PRIORITY

This patent application claims priority to U.S. provisional patent application No. 63/308,044, titled “DYNAMICALLY RIGIDIZING COMPOSITE MEDICAL STRUCTURES,” filed on Feb. 8, 2022, and herein incorporated by reference in its entirety.

### INCORPORATION BY REFERENCE

All publications and patent applications mentioned in this specification are herein incorporated by reference in their entirety to the same extent as if each individual publication or patent application was specifically and individually indicated to be incorporated by reference.

### BACKGROUND

There are a multitude of types of elongate tubular medical devices that are inserted into openings (natural or made) in the body for diagnosis and therapy. For example, these devices may include endoscopes, catheters, wires, sheaths, overtubes, cannulas, and trocars. These devices may include both hand tools, and robotically operated systems. These devices are typically available in forms that are both flexible and rigid. There are both advantages and disadvantages to flexible devices and to rigid devices. Advantageously, virtually every major organ in the human body can be reached with a flexible catheter or endoscope. However, flexible endoscopes and catheters rely on reaction forces generated by pushing against the tissue of the body cavity being explored to navigate around corners or bends in the anatomy. Flexibility may be problematic when navigating through body regions having highly tortuous passages, areas that are comparatively open, friable elements that are easily dislodged, or passages of varying (or large) luminal diameter, where it may be difficult to make reliable contact with the outer diameter of the tube. Further, highly flexible tubes may distort, buckle, prolapse, loop, or may have trouble supporting additional tools or devices. Flexible devices make precision tip motion difficult, they can highly compromise precision motion and control, and do not support procedures that require high tip loads. These issues can lead to extended procedure times, trauma, complications, and damage-including death. Rigid devices may create their own challenges. Rigid devices can be difficult to navigate within the body and are simply unable to navigate to a wide variety of locations. Advancing them can cause significant anatomical distortion, trauma, complications, and damage-including death. Under certain circumstances, both flexible and rigid devices can cause highly compromised clinical outcomes.

Thus, it may be beneficial to provide devices that are selectively rigidizable, and which may controllably transition between highly flexible and highly rigid configurations. Of particular interest are rigidizable tubes descried in U.S. Pat. No. 11,135,398, titled “DYNAMICALLY RIGIDIZING COMPOSITE MEDICAL STRUCTURES”, which is herein incorporated by reference in its entirely. Although such tools may provide safe, efficient, and precise access to otherwise difficult to reach anatomical locations, it would be beneficial to modify all or certain regions of these devices that enable improved trackability, enhanced flexibility or stiffness, reduced shape distortion, enhanced safety, ease of manufacture, and/or increased reliability.

Described herein are apparatuses and methods that may address these needs.

### SUMMARY OF THE DISCLOSURE

Described herein are rigidizing apparatuses (e.g., devices, system, etc.) as well as methods of using them. For example, described herein are rigidizing devices including coil-wound tubes, also referred to herein as inner and/or outer coil-wound tubes (e.g., ICWTs or OCWTs) that include a tube of matrix material onto which one or more coil supports is wound. The coil support may be formed of a wire, cable, etc., including a ribbon of material. In some examples the coil-wound tube may include matrix material sandwiched between a first support wire coil region (e.g., helical coil) and a second support wire coil region (e.g., helical coil) in which the first and second support wire coil regions are part of the same component, such as a wire, that doubles back over itself as a single, continuous piece (e.g., a single continuous filament, ribbon, wire, etc.). For example, the first support wire coil region may be referred to as a proximal-to-distal winding which may double back over (or in some examples, under) a distal-to-proximal winding forming the second support wire coil region.

In any of the apparatuses (e.g., devices, systems, etc.) described herein, the first and/or the second support wire coli regions are continuous where they meet but are not continuous as they extend along the proximal-to-distal length of the apparatus. For example, the support coil may extend only partially down the length of the elongate rigidizing apparatus (e.g., over the distal end) and does not extend down the full length of the device. In some examples the starting end of the first support wire coil region starts more distally than the distal end region of the second support wire coil, or vice versa.

The first and second coil regions may be wound in the same direction, or they may be counterwound. The pitches of the distal-to-proximal winding may be the same as the proximal-to-distal winding, or they may be different. In some variations the pitch of the distal-to-proximal winding may be uniform, or it may vary; the pitch of the proximal-to-distal winding may be uniform, or it may vary. The coils may be made of the same material, or of different materials. The coils may have the same cross-sectional geometry, or different cross-sectional geometry. A continuous wind allows the user to manufacture the tube with a single piece of wire, which may have manufacturing advantages. A continuous wind creates a distal or proximal region with no cut wires, which eliminates the risks associated with wires splaying outwards or inwards, which can cause geometrical distortions to the catheter body or tip and could cause trauma to the patient or user.

Any of the apparatuses described herein may be configured as components that are utilized for rigidizing apparatuses. These can be used for negative or vacuum based systems, or for higher pressure systems (i.e., for more than one atmosphere, including for positive pressure systems). The coiled regions may be used for systems in which the coiled regions received external pressure, which creates forces trying to crush the tube. The coiled regions may be used for systems in which the coiled regions receive vacuum consolidation forces.

Any of the apparatuses described herein may be configured as rigidizing apparatuses that are rigidizing by inflation of one or more bladder regions; the bladder regions of these apparatuses (either or both sides of the bladder's surface) may be configured to prevent sticking or other failure modes by including an additive (e.g., lubricant, powder etc.) and/or a texture, and/or a coating. For example, the bladder region may include a lubricious material such as glycerin. In some examples the bladder region may be inflated with a material to reduce sticking. For example, the bladder region may be inflated with a lubricious material.

Any of the apparatuses described herein may be configured as rigidizing apparatuses that are formed of a plurality of layers (including radially arranged layers) that are configured to prevent delamination of the layers by including one or more bonding layers or tie layers. A tie layer may include a layer that serves as an effective intermediary layer that may be particularly beneficial for bonding. For example, if layer A would not bond to layer C, a tile layer B may be used by having layer A bonding to layer B, layer B bond to layer C, and therefore effectively bonding layer A to layer C. The tie layer may be significantly thinner and may be continuous or discontinuous.

Any of the apparatuses described herein may be configured as rigidizing apparatuses configured for enhanced tracking by reducing tip stiffness. These apparatuses may be configured to adjust or tune the flexibility of the distal tip by setting the spacing of various layers at the distal tip region.

Any of the apparatuses described herein may be configured as rigidizing apparatuses that are rigidizing by inflation from a distal (or intermedial) end region rather than strictly or exclusively from the proximal end region.

Any of the apparatuses and methods described herein may be configured for use as a positionable stable platform that may be used as an imaging landmark, computational landmark, or launching point for one or more medical procedures.

For example, described herein are catheter devices including an elongate flexible tube comprising: an intermediate matrix layer; a first reinforcing coil extending from a proximal end to a distal end against an outer surface of the intermediate matrix layer; a second reinforcing coil extending from the proximal end to the distal end against an inner surface of the intermediate matrix layer, wherein the first reinforcing coil and the second reinforcing coil form a continuous length of material that doubles over itself at the distal end of the intermediate matrix layer; and an elastomeric covering over the second reinforcing coil and extending from the distal end to the proximal end. In any of these devices, one or more outer matrix layers may be positioned between the first reinforcing coil and the intermediate matrix layer and/or between the second reinforcing coil and the intermediate matrix layer. The catheter device may be configured to be pressurized so as to rigidize the catheter. The first reinforcing coil and the second reinforcing coil may comprise a wire. The wire may comprise a ribbon (e.g., a flat wire) in which at least two surfaces are substantially parallel to each other. The elastomeric covering may be fused to the intermediate matrix and over the second reinforcing coil.

Any of these apparatuses may include a second elastomeric covering fused to the intermediate matrix over the first reinforcing coil.

Any of these apparatuses may be configured as rigidizing catheters or rigidizing members. For example these apparatuses may include a braid layer positioned over the elongate flexible tube, an outer layer over the braid layer, an inlet between the elongate flexible tube and the outer layer and configured to attach to a source of vacuum or pressure, wherein the catheter device is configured to have a rigid configuration when vacuum or pressure is applied through the inlet (e.g., a pressure inlet or pressure port) and a flexible configuration when vacuum or pressure is not applied through the inlet.

Also described herein are rigidizing catheters, comprising: an elongate member comprising a plurality of layers; a bladder layer among the plurality of layers, wherein the rigidizing catheter comprises features to prevent adhesion of the bladder layer to itself or surrounding layers, wherein the rigidizing catheter is configured to be pressurized to provide rigidity to the catheter.

The features may include texturing (e.g., a surface texture on an outer or inner surface of the bladder layer, and/or in some cases on an adjacent surface). For example, the features may comprise texturing of surrounding layers. Alternatively or additionally, the features may comprise an additive, such as a colorant, mineral, wax, or lubricant. In some examples the features comprise a coating and/or a powder. For example, the features may comprise a braid layer adjacent to the bladder layer. The features may comprise a channel or tube extending along the bladder layer.

Also described herein are rigidizing catheters including: an elongate member comprising a plurality of layers, comprising an outer layer; an inner layer laminated to the outer layer; a reinforcement layer positioned between the outer layer and the inner layer; a tie layer positioned between the outer layer and the inner layer and spanning a reinforcement layer, wherein the outer layer is laminated to the inner layer through the tie layer, wherein the rigidizing catheter is configured to be pressurized to provide rigidity to the catheter. The reinforcement layer may comprise a wire.

A rigidizing catheter may include: an elongate member comprising a plurality of layers including an outer layer and an inner layer, wherein a distal end of the outer layer is axially distanced from a distal end of the inner layer by a distance of at least about 2 mm (e.g., about 1.8 mm, 1.7 mm, 1.5 mm, 1.4 mm, 1.3 mm, 1.2 mm, 1.1 mm, 1 mm, 0.9 mm, 0.8 mm, 0.7 mm, 0.6 mm, 0.5 mm, etc.), or, as the catheter shaft diameter scales, greater than 0.25 fold of the diameter of the catheter, further wherein the rigidizing catheter is configured to be pressurized to convert the catheter from an unpressurized and flexible configuration into a pressurized and rigid configuration.

In any of these examples, the outer layer may comprise a reinforcement layer comprising a wire. The inner layer may comprise an elongate flexible tube. The elongate flexible tube may be configured as described above.

For example, a rigidizing catheter may include: an elongate member comprising a plurality of layers including a bladder layer; an inflation lumen extending between two of the layers and to a distal end of the elongate member, wherein the inflation lumen is configured to provide pressure to the bladder layer from a distal end of the rigidizing catheter, further wherein the rigidizing catheter is configured to be pressurized to convert the catheter from an unpressurized and flexible configuration into a pressurized and rigid configuration. The inflation lumen may include a layflat tube. The inflation lumen may comprise nylon, polyethylene, thermoplastic polyurethane (tpu), or polyethylene terephthalate (e.g., PET). The inflation lumen may comprise a heat shrink tubing. The inflation lumen may have a wall thickness of about 0.0005″ thick. In some examples the inflation lumen comprises a one-way valve configured to allow application of vacuum and not pressure. The inflation lumen may comprise a breather mechanism, such that it continues to pass vacuum and does not seal itself off while under negative pressure.

Also described herein are methods of treating a patient using the rigidized apparatus as a stable platform for performing one or more additional procedures. For example, the method may include: inserting a rigidizing catheter into a body lumen of a patient in a flexible configuration; rigidizing the rigidizing catheter; visualizing a distal rigid end region of the rigidizing catheter; using the distal end of the rigidized rigidizing catheter as a visual indicator, a datum from which subsequent directives are computationally derived, and as a stable base for performing one or more procedures within the patient.

A rigidizing device may include: an elongate flexible tube; a braid layer positioned over the elongate flexible tube; a plurality of particles surrounding the braid layer; an outer layer over the flexible tube, the braid layer, and plurality of particles; and an inlet between the elongate flexible tube and the outer layer and configured to attach to a source of vacuum or pressure;

wherein the rigidizing device is configured to have a rigid configuration when vacuum or pressure is applied through the inlet and a flexible configuration when vacuum or pressure is not applied through the inlet.

A rigidizing device may include: an elongate member comprising a plurality of layers comprising; an inner layer; a plurality of particles positioned over the inner layer with particles potentially amidst a rigidizing layer; an outer layer over the inner layer and plurality of particles; and an inlet between the inner layer and the outer layer and configured to attach to a source of vacuum or pressure; wherein the rigidizing device is configured to have a rigid configuration when vacuum or pressure is applied through the inlet and a flexible configuration when vacuum or pressure is not applied through the inlet.

An elongate rigidizing device having a plurality of layers may include; an inner layer comprising an elongate flexible tube; a braid layer positioned over the elongate flexible tube along a first portion of a length of the elongate member; a plurality of particles surrounding the braid layer along a second portion of a length of the elongate member; an outer layer over the inner layer, the braid layer, and plurality of particles; and an inlet between the inner layer and the outer layer and configured to attach to a source of vacuum or pressure; wherein the rigidizing device is configured to have a rigid configuration when vacuum or pressure is applied through the inlet and a flexible configuration when vacuum or pressure is not applied through the inlet. The first portion may include a braid layer in addition to a plurality of particles. The second portion may comprise a plurality of particles without a braid layer. The plurality of particles may comprise a powder. The particles may serve to augment the stiffness provided by the braid system, as the particles ‘jam’ when consolidated. The particles may serve to augment the stiffness provided by the braid system, as the particles interdigitate between braid elements when consolidated. The properties of the plurality of particles may vary along a length of the elongate member, resulting in varying stiffnesses along the elongate member when the device is in the rigid configuration.

For example, described herein are devices, the devices including an elongate flexible tube comprising: an intermediate matrix layer; a reinforcing coil comprising an outer reinforcing coil region extending from a first proximal region to a second distal region and a second reinforcing coil region extending from the second distal region to the first proximal region adjacent to the intermediate matrix layer, wherein the outer reinforcing coil region and the inner reinforcing coil region form a continuous length; and an outer layer.

Any of these devices may include a pressure inlet in fluid communication with a gap region between one or more of the layers. In particular, these rigidizing devices may include a pressurizing gap region between the reinforced layer (e.g., the intermediate matrix layer and the intermediate or outer matrix layer(s)) and a bladder layer that is configured to receive either positive pressure or negative pressure to rigidize the device. The intermediate matrix layer may be between the outer reinforcing coil region and the inner reinforcing coil region.

These devices may include an elastomeric covering over the inner reinforcing coil and extending from the first proximal region to the second distal region. A helical angle of the inner reinforcing coil region may have a different magnitude and an opposite direction than a helical angle of the outer reinforcing coil region. A helical angle of the inner reinforcing coil region may have the same magnitude but an opposite direction than a helical angle of the outer reinforcing coil region. A pitch of the inner reinforcing coil region may be different from a pitch of the outer reinforcing coil region. A transverse cross-section of the inner reinforcing coil region may be different than a transverse cross-section of the outer reinforcing coil region.

In any of these devices, the reinforcing coil may comprise a wire. For example the reinforcing coil may comprise a ribbon having two sides that are substantially parallel. The elastomeric covering may be fused to the intermediate matrix over the second reinforcing coil. In some examples the device includes a braid layer adjacent to the outer layer. In some examples the braid layer is adjacent to the inner layer. In general, a rigidizing device (which may include or be part of a rigidizing apparatus) may have a wall thickness that includes an inner reinforced layer (e.g., an ‘inner coil-wound tube’) a bladder layer, a variable stiffness layer (e.g., a braid layer, a knitted layer, a woven layer, etc. which may comprise a plurality of lengths of one or more filaments that cross over each other), an air gap and an outer reinforced layer (e.g., an outer coil-wound tube). The device may be configured to have a rigid configuration when either positive pressure or negative pressure is maintained through the pressure inlet and a flexible configuration when positive pressure or negative pressure is not maintained through the inlet. The pressure inlet is in fluid communication with the bladder layer. The air gap may be in fluid communication with one or more vents, e.g., at a proximal end of the device. As used herein, the terms fiber, filament and strand may be used equivalently. A fiber may be made up of one filament (e.g., monofilament) or more than one filament.

For example, described herein are rigidizing devices that include an elongate flexible tube having a wall thickness comprising one or more reinforced layers each including an intermediate matrix layer; a reinforcing coil comprising an outer reinforcing coil region that is continuous with an inner reinforcing coil region; and an outer matrix layer. The device is configured to have a rigid configuration when a positive or a negative pressure is maintained within the wall thickness and a flexible configuration when vacuum or pressure is not maintained within the wall thickness.

As mentioned, described herein are devices including non-stick or anti-stick inflatable bladders for rigidizing the device. For example, a rigidizing device may include: an elongate member comprising a plurality of layers; a bladder layer among the plurality of layers, wherein the rigidizing device comprises one or more features to prevent adhesion of the bladder layer to itself or surrounding layers, wherein the rigidizing device is configured to be pressurized to rigidize. The one or more features may comprise one or more of: texturing, an additive, texturing of surrounding layers, a lubricant, particulates, a coating, a braid layer adjacent to the bladder layer, and/or a channel or tube extending along the bladder layer.

Also described herein are rigidizing devices including one or more tie layers. For example, any of the devices described herein may include: an elongate member comprising a plurality of layers, comprising: an outer layer; an inner layer laminated to the outer layer; a reinforcement layer positioned between the outer layer and the inner layer; a tie layer positioned between the outer layer and the inner layer and spanning a reinforcement layer, wherein the outer layer is laminated to the inner layer through the tie layer, wherein the rigidizing device is configured to be pressurized to rigidize. The reinforcement layer may comprise a wire.

Also described herein are rigidizing devices formed of multiple layers, that are

adapted to have a narrow or smaller profile. For example, any of the devices described herein may include: an elongate member comprising a plurality of layers including an outer layer and an inner variable stiffness layer (e.g., braided layer, knitted layer, woven layer), wherein a distal end of the outer layer is axially separated from a distal end of the inner variable stiffness layer by an axial distance of at least about one quarter of a diameter (radial distance) of the device transverse to the elongate member, further wherein the rigidizing device is configured to be pressurized to convert from an unpressurized and flexible configuration into a pressurized and rigid configuration. The distal end of the outer layer may be separated from the distal end of the inner variable stiffness layer by at least 2 mm. The outer layer may comprise a reinforcement layer comprising a wire. The inner layer may comprise an elongate flexible tube. The elongate member may further include a second inner layer, wherein a distal end of the second inner is between the distal end of the outer layer and the distal end of the inner layer. The second inner layer may comprise a helically counter-wound reinforcing coil.

Also described herein are rigidizing devices that are inflatable to rigidize, and that distribute the pressure for rigidization at various points or along the length of the device. For example, these apparatuses may include: an elongate member comprising a plurality of layers including a bladder layer; an inflation lumen extending between two of the layers and to a distal end region of the elongate member, wherein the inflation lumen is configured to provide pressure to the bladder layer from a distal end of the rigidizing device, further wherein the elongate member configured to be pressurized to convert from an unpressurized and flexible configuration into a pressurized and rigid configuration. Similarly, these apparatuses can be used to enhance distal vacuum, when used as vacuum transmission conduits, for vacuum rigidizing devices. In some examples these devices can utilize both negative and positive pressure in combination (on either sides of a bladder) for enhanced rigidization. The inflation lumen may comprise a layflat tube. The inflation lumen may comprise PET. The inflation lumen may comprise heat shrink tubing. The inflation lumen may comprise a wall thickness of about 0.0005″ thick. The inflation lumen may comprise a one way valve configured to allow application of vacuum and not pressure. The inflation lumen may comprise breather mechanism configured to keep the inflation lumen open during application of vacuum.

Also described herein are method of using any of these devices as a datum or reference. For example, a method of treating a patient may include: inserting a rigidizing device into a body lumen of a patient in a flexible configuration, wherein the rigidizing device extends an entire length from a handle to a distal end region; rigidizing a portion or the entire length of the rigidizing device by applying positive or negative pressure; visualizing the distal rigid end region of the rigidizing device; using the distal end region of the rigidized rigidizing device as a stable locational datum for performing one or more procedures within the patient. Rigidizing the device may comprise applying positive pressure. Rigidizing the device may comprise applying negative pressure. Any of these methods may include making one or more measurements from the stable locational datum.

Any of the devices described herein may include particles in addition to a rigidization layer or a braid to rigidize the device. For example, a rigidizing device may include: an elongate flexible tube; a braid layer positioned over the elongate flexible tube; a plurality of particles surrounding the braid layer; an outer layer over the flexible tube, the braid layer, and plurality of particles; and an inlet between the elongate flexible tube and the outer layer and configured to attach to a source of vacuum or pressure; wherein the rigidizing device is configured to have a rigid configuration when vacuum or pressure is applied through the inlet and a flexible configuration when vacuum or pressure is not applied through the inlet, further wherein in the rigid configuration the plurality of particles enhance rigidity by compacting within the braid layer.

For example, an elongate rigidizing device having a plurality of layers, the device comprising; an inner layer comprising an elongate flexible tube; a braid layer positioned over the elongate flexible tube along a first portion of a length of the elongate member; a plurality of particles surrounding the braid layer along a second portion of a length of the elongate member; an outer layer over the inner layer, the braid layer, and plurality of particles; and an inlet between the inner layer and the outer layer and configured to attach to a source of vacuum or pressure; wherein the rigidizing device is configured to have a rigid configuration when vacuum or pressure is applied through the inlet and a flexible configuration when vacuum or pressure is not applied through the inlet. The first portion may comprise a braid layer in addition to a plurality of particles. The second portion may comprise a plurality of particles without a braid layer. The plurality of particles may include a powder or particles of a specific size. The plurality of particles may vary along a length of the elongate member, resulting in varying stiffnesses along the elongate member when the device is in the rigid configuration.

All of the methods and apparatuses described herein, in any combination, are herein contemplated and can be used to achieve the benefits as described herein.

### BRIEF DESCRIPTION OF THE DRAWINGS

A better understanding of the features and advantages of the methods and apparatuses described herein will be obtained by reference to the following detailed description that sets forth illustrative embodiments, and the accompanying drawings of which:

FIG. 1A shows one example of a rigidizing device.

FIG. 1B schematically illustrates an example of a robotic apparatus including a pair of nested rigidizing members.

FIG. 1C schematically illustrates an example of a robotic apparatus including a pair of nested rigidizing members.

FIGS. 1D-1H illustrate examples of pluralities of strand lengths that cross each other which may form a variable stiffness layer of any of the rigidizing apparatuses described herein. FIG. 1D shows a knitted variable stiffness layer having a weft knit pattern, FIG. 1E shows an example of a knitted variable stiffness layer having a warp knit pattern. FIGS. 1F shows an example of a braided variable stiffness layer and FIG. 1G shows an example of a discontinuous braided variable stiffness layer. FIG. 1H shows an example of a woven rigidizing layer formed of filament; this woven rigidizing layer may be used as part of a rigidizable device as described herein.

FIGS. 2A-2B show an example of a portion of a vacuum rigidizing apparatus as described herein. FIG. 2A shows a section through the exemplary vacuum rigidizing member of the apparatus. FIG. 2B shows an enlarged view of a portion of the section, illustrating the arrangement of layers in the un-rigidized configuration.

FIGS. 3A-3B show an exemplary pressure rigidizing apparatus. FIG. 3A shows a longitudinal section through the pressure rigidizing apparatus. FIG. 3B shows a transverse section through the pressure rigidizing apparatus.

FIG. 4A is a schematic section through an example of an inner coil-wound tube (ICWT) as described herein. FIGS. 4B and 4C show examples of reinforcement wires of an ICWT similar to that shown in FIG. 4A.

FIGS. 5A-5C illustrate examples of inner coil-wound tubes having different coil angles.

FIG. 6 schematically illustrates a section through one example of a pressure rigidizing apparatus.

FIG. 7A shows an example of a section trough a rigidizing device as described herein.

FIG. 7B shows an example of an inner layer of an outer rigidizing device comprising one or more tie layers.

FIG. 7C shows an example of an inner layer of an inner rigidizing device comprising one or more tie layers.

FIGS. 8A and 8B show a cross sectional view of a rigidizing device having at least two layers with separated distal ends.

FIG. 8C shows a plot of stiffness vs. length of the rigidizing device of FIGS. 8A and 8B.

FIG. 9 shows an example of a rigidizing device comprising an inflation lumen configured to inflate a bladder layer at a distal end of the device.

FIG. 10 shows an example of a method for performing a medical procedure in the pulmonary vasculature using a rigidizing device as a stabilizing and reference or guide base, or computational guide.

FIG. 11 shows an example of a rigidizing device comprising a braid layer and a plurality of particles near the braid layer for the bladder layer to press against when rigidized.

FIG. 12 shows an example of a rigidizing device comprising a plurality of particles for the bladder layer to press against when rigidized.

FIG. 13 shows an example of a rigidizing device comprising a plurality of particles in a first section and a braid layer in a second section of the rigidizing device.

FIG. 14 shows an example of a rigidizing device comprising a plurality of particles and a braid layer in a first section and just a braid layer in a second section of the rigidizing device.

FIGS. 15A-15B and 15C-15D schematically illustrate examples of the distal end regions of rigidizing devices having variable pitch coil wound tubes for enhanced flexibility. FIGS. 15A and 15C show perspective views and FIGS. 15B and 15C show side views.

FIGS. 16A-16E illustrate examples of portions of a distal end region of a rigidizing device. FIG. 16A schematically illustrates an example of a tip region sealing between an inner region (e.g., inner coil wound tube) and an outer region (e.g., outer coil wound tube). FIG. 16B shows one example of a reflowed polymeric tip region that is configured to provide a smooth profile at the distal tip region. FIG. 16C schematically illustrates an example of a method of forming a rolled-back reflowed polymeric tip region such as that shown in FIG. 16B. FIG. 16D illustrates an example of a distal end region of a portion of a rigidizing device having a funnel-shaped reflowed polymeric tip sealing coupling the inner region to the outer region. FIG. 16E illustrates an example of a distal end region of a portion of a rigidizing device having a r olled-back reflowed polymeric tip sealing coupling the inner region to the outer region similar to that shown in FIG. 16B.

FIG. 17 schematically illustrates a section through an example of a distal end region of a rigidizing device as described herein.

FIG. 18 schematically illustrates an example of a section through an example of a proximal end of a rigidizing device as described herein.

FIG. 19 schematically illustrates an example of the distal end region of an apparatus in which the lower coil of the inner coil wound tube is radially re-directed at the distal end region to enhance flexibility and prevent disruption of the distal end.

FIG. 20 schematically illustrates an example of the distal end region of a rigidizing device in which the bladder (e.g., pressure bladder) has a thicker distal end region within the layered structure.

FIG. 21A schematically illustrates one example of a pair of nested rigidizing members in which the outer rigidizing member is configured so that that the distal end region bends without compressing the sides (e.g., “parallelogram” bending). FIG. 21B schematically illustrates parallelogram bending; FIG. 21C schematically illustrates compressive bending. FIGS. 21D1 and 21D2 illustrate bending without compressing the lengths of the sides. FIG. 21E shows an example of a portion of a rigidizing member tracking over a curved member while a region of bends by parallelogram bending. FIG. 21F schematically illustrates one example of an apparatus configured to bend by parallelogram bending. In this configuration, the ICWT has dual wire reinforcement, and one of the wires extends beyond the other wire. FIG. 21G is a section through an apparatus configured to bend by parallelogram bending.

FIGS. 22A-22C schematically illustrate one example of an axial accommodator configured to couple to the proximal end of a rigidizing member to relieve axial stresses when rigidizing the member. FIG. 22A shows an example of a section through an axial accommodator. FIG. 22B shows a perspective view of the section through the axial accommodator of FIG. 22A. FIG. 22C is a perspective view of the axial accommodator of FIGS. 22A and 22B.

### DETAILED DESCRIPTION

In general, described herein are rigidizing apparatuses (e.g., devices, system, etc.) that may be configured as, or to aid in the transport of, a catheter or scope (e.g., endoscope) or other medical instrument through a curved or looped portion of the body, e.g., a portion of the bodily lumen, including the gastrointestinal tract (upper, lower (colon), middle (small intestine), and ducts (biliary and pancreatic)), the urological tract, the pulmonary vasculature, the venous and arterial vasculature, and the neuro vasculature, as well as methods of using them. In particular, described herein are rigidizing devices including one or more reinforced layers formed as coil-wound tubes. For example, these rigidizing devices may include an inner coil-wound tube (ICWT) and/or an outer coil-wound tube (OCWT). In some examples the coil-wound tube includes oppositely-wound coils, wire or ribbons that are counterwind over an intermediate layer (e.g., in some examples a tie layer), in which both the outer wound coil and the inner wound coil are formed of a single, continuous piece (e.g., filament, wire, etc.). For example, an inner coil-wound tube may include multiple layers, and may itself form a layer of the wall diameter of the rigidizing device. The multiple layers of the inner coil-wound tube may include an intermediate matrix layer, a pair of oppositely-wound coils, which may be arranged to provide unique attributes. The reinforced inner coil wound tube may be particularly helpful in variations in which the inner coil wound tube resists the pressure applied to rigidizing the variable stiffness layer, which may be externally pressurized by applying pressure to drive a bladder layer against the variable stiffness layer.

Any of the apparatuses described herein may be configured as rigidizing apparatuses that are rigidizing by negative pressure (vacuum) or positive pressure and/or by inflation or deflation of one or more bladder regions. The bladder regions of these apparatuses may be configured to prevent sticking or other failure modes by including an additive (e.g., lubricant, etc.) and/or a texture (e.g., grooves, channels, ridges, etc.), and/or a coating (e.g., lubricious coating, hydrophilic coating, hydrophobic coating, etc.) and or particles or particulates.

Any of the apparatuses described herein may be configured as rigidizing apparatuses that are formed of a plurality of layers (including radially arranged layers) that are configured to prevent delamination of the layers by including one or more bonding layers or tie layers. As used herein a tie layer may form a layer that is ties other layers together. For example, if layer A does not bond well to layer C, the system can be configured so that layer A may bond to layer B, and layer B might bond to layer C, so layer B may serve to ‘tie’ layer A to layer C.

Any of the apparatuses described herein may be configured as rigidizing apparatuses configured for enhanced tracking by reducing tip stiffness. These apparatuses may be configured to adjust or tune the flexibility of the distal tip by setting the axial and radial spacing of various layers at the distal tip region. Tip flexibility and trackability may be important performance characteristics for a device, including a catheter or endoscope. This could be particularly useful for nested devices, such as mother-daughter devices, or endoscopes within overtubes, or catheters within catheters. In general, apparatuses having a rigid tip typically do not track well.

Having multiple layers terminate at the same distal axial location often serves to create rigid tips. By axially spacing the distal termination of layers, the distal tip region flexibility may be modulated for enhanced trackability. These axial offset distances can be a set distance (for example, 2 or 3 mm), or they can be a percentage of the diameter of the catheter (for example, a quarter the catheter's diameter, half the catheter's diameter, 1 × the catheter's diameter, 2× the catheter's diameter, etc.).

Any of the apparatuses described herein may be configured as rigidizing apparatuses that are rigidizing by inflation from a distal (or any intermediate point) end region rather than strictly or exclusively from the proximal end region.

Any of the apparatuses and methods described herein may be configured for use as a positionable stable platform having a tip that may be used as an imaging landmark, computational landmark, (ultrasound, CT, fluoroscopy, MRI, echo) or launching point for one or more medical procedures. The imaging landmark may be due to density (for example, a gold, tungsten, tantalum, platinum, osmium, or iridium ring) and/or magnetic properties (e.g., such as an embedded magnetic element). This can create a datum from which subsequent procedural computational dictates can be derived.

In general, any of the apparatuses and methods described herein may be used in combination with any of these features and methods of using them. For example, the same apparatus may include an ICWTs that includes oppositely-wound layers ribbons or wire sandwiched over an intermediate (e.g., tie) layer in which both the outer wound coil and the inner wound coil are formed of a single, continuous piece, and/or may include a plurality of layers (including radially arranged layers) that are configured to prevent delamination of the layers by including one or more bonding layers or tie layers, and/or may include a bladder region configured to prevent sticking or other failure modes by including an additive (e.g., lubricant, etc.) and/or a texture, and/or a coating.

The rigidizing devices described herein can be long, thin, and hollow and can transition quickly from a flexible configuration (i.e., one that is relaxed, limp, or floppy) to a rigid configuration (i.e., one that is stiff and/or holds the shape it is in when it is rigidized). In some examples the rigidizing apparatus may include a plurality of layers (e.g., coiled or reinforced layers, slip layers, variable stiffness layer, bladder layers and/or sealing sheaths) can together form the wall of the rigidizing devices. The rigidizing devices can transition from the flexible configuration to the rigid configuration, for example, by applying a vacuum or pressure to the wall of the rigidizing device or within the wall of the rigidizing device. With the vacuum or pressure removed, the layers can easily shear or move relative to each other. With the vacuum or pressure applied, the layers can transition to a condition in which they exhibit substantially enhanced ability to resist shear, movement, bending, torque and buckling, thereby providing system rigidization.

The examples of rigidizing apparatuses described herein may use pressure (positive pressure) and/or negative pressure to selectively and controllable rigidize and may be adapted to perform reductions as described herein. The apparatuses described herein may use one or both vacuum pressure (negative pressure) and positive pressure, which may be applied, and sometimes simultaneously applied, the rigidizing device to rigidize and in some variations to de-rigidize the device. Positive and/or negative pressure may be applied on one or both sides of bladder layer to rigidize and/or de-rigidize. In some examples, positive and/or negative pressure may be simultaneously applied to different regions of the device and its various layers and cross-sections. The features and methods described herein are not limited to pressure (positive or negative) rigidizing apparatuses, however; the method described herein may be used with any appropriate rigidizing apparatus.

The rigidizing (e.g., selectively rigidizing) apparatuses described herein can provide rigidization for a variety of medical applications, including catheters, sheaths, scopes (e.g., endoscopes), wires, overtubes, trocars or laparoscopic instruments. The rigidizing devices can function as a separate add-on device or can be integrated into the body of catheters, sheaths, scopes, wires, or laparoscopic instruments. The devices described herein can also provide rigidization for non-medical structures. The rigidizing devices described herein may also be non-tubular (including planar) rigidizing structures.

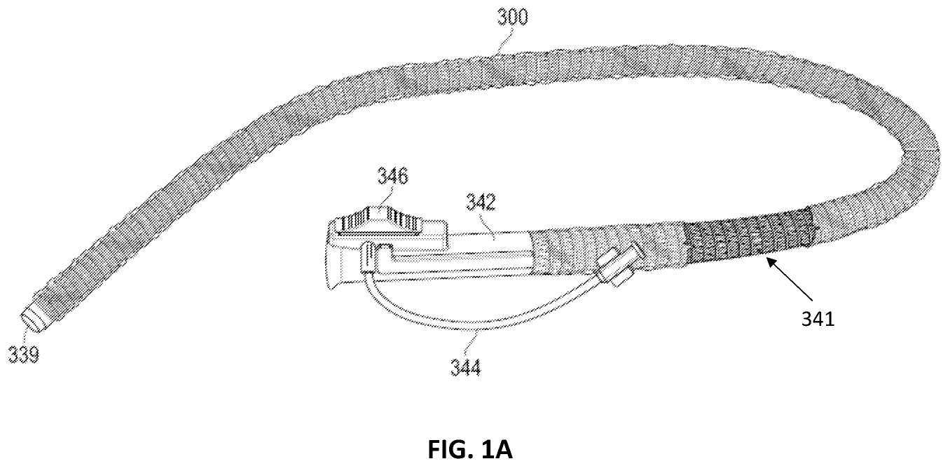

An exemplary rigidizing apparatus is shown in FIG. 1A. The system shown includes a rigidizing device 300 having a wall with a plurality of layers including a variable stiffness layer (e.g., a braided layer, a knitted layer, a woven layer, etc.), an outer layer (part of which is cut away 341 in this example to show the braid thereunder), and an inner layer. The inner layer may be an inner coil-wound tube (ICWT). In general, any of these apparatuses may include a leak-proof ICWT that may resist radial compression while providing a consistent circumferential cross section. Any of these apparatuses may further include a proximal end that may be configured as a handle 342 having a vacuum or pressure inlet 344 (generically referred to as a pressure inlet) to supply vacuum (negative pressure) or positive pressure to the rigidize the device 300. An actuation element 346 (e.g., control, such as a switch, button, etc.) can be used to turn the vacuum or pressure on and off to thereby transition the rigidizing device 300 between flexible and rigid configurations. The distal end 339 (including a distal end region) of the rigidizing device 300 can be smooth, flexible, and atraumatic to facilitate distal movement of the rigidizing device 300 through the body. Further, the apparatus (including the tip 339) can taper from the distal end to the proximal end to further facilitate distal movement of the rigidizing device 300 through the body. In this example, the rigidizing apparatus is configured as an overtube, but other configurations may be used. The rigidizing device may have a constant inner or outer diameter over its length, or it may taper, or it may have local zones of varying inner or outer diameter.

In general, any of these apparatuses may be configured as robotic apparatuses that include one or more (e.g., a pair of nested) rigidizing members. FIG. 1B schematically illustrates one example of a robotic apparatus including a pair of rigidizing members that are nested and may be moved (e.g., telescopically) relative to each other. For example, in FIG. 1B the inner rigidizing member 3005 may steerable and move within the inner lumen of the outer rigidizing member 303; the inner rigidizing member 305 is nested within the outer rigidizing member 303, and the inner and outer rigidizing members may move axially and rotationally relative to each other. The apparatus shown in FIG. 1B also includes a robotic controller 307 that is configured to couple to the proximal ends of the inner and outer rigidizing member and to drive movement (axial and/or rotational movement) of each of the inner and outer rigidizing members. The robotic controller may include circuitry, such as control circuitry, feedback circuitry, pressure control circuitry, etc. one or more actuators (e.g., motors), and inputs (e.g., keyboards, buttons, etc.) for controlling and coordinating the movement of the inner and outer rigidizing members. The controller may include or may be coupled to the source of positive and/or negative pressure. The robotic controller may be configured to advance and retract the apparatus in a controlled pathway by selectively and alternately rigidizing and de-rigidizing the inner and/or outer rigidizing members while moving the inner and outer rigidizing members relative to each other (e.g., advancing and/or retracing) to set the pathway of the nested device, as illustrated in FIG. 1B.

FIG. 1C illustrates another example of a robotic apparatuses also comprising a nested pair of outer and inner rigidizing members. In FIG. 1C details of one example of a robotic controller configured to control operation of the robot are shown. In this exemplary apparatus 9300z, the outer rigidizing member 9300 and the inner rigidizing member 9310 may be terminated together in the robotic controller. In some examples the robotic controller may be configured as a cassette 9357 that may engage with a separate robotic driver (not shown in FIG. 1C) that may actuate the steering (e.g., of a distal end region of one or both the inner and/or outer rigidizing member), advancing and/or rotating of either or both the inner and/or outer rigidizing members.

In FIG. 1C, the outer rigidizing device 9300 can be movable with respect to the inner rigidizing device 9310 by rotation of a disk 9389 that is mounted to the cassette 9357. For example, the disk 9389 can be a pinion, and the outer rigidizing device 9300 may have a rack 9382 including a plurality of small teeth on the outside thereof. Rotating the disk 9389 against teeth 9382 may cause outer rigidizing device 9300 to advance forward or backward relative to the inner rigidizing device 9310. In some examples, the possible movement or translation of the rigidizing devices 9300, 9310 is limited by the size or design of the cassette 9357.

The cassette 9357 can further include additional disks 9371a, 9371b that may connect to cables 9363a,b respectively, to steer (e.g., bend or deflect) the tip of the inner rigidizing device 9310 (and/or outer rigidizing device 9300). Other steering mechanisms (e.g., pneumatics, hydraulics, shape memory alloys, EAP (electro-active polymers), or motors) are also possible. Again, in examples with different steering mechanisms, one or more disks in the cassette 9357 (e.g., disks 9371a, 9371b) may be used to actuate the steering.

The cassette 9357 can further include bellows 9303a, 9303b that may connect to the pressure gap of the inner rigidizing device 9310 and the outer rigidizing device 9300, respectively. In this example compressing bellows 9303a, 9303b may drive fluid through pressure lines 9305z, causing the pressure in the pressure gap of the inner rigidizing devices 9310, 9300 to rise, causing the rigidizing devices 9310, 9300 to become rigid. Activation of the bellows 9303a, 9303b may be applied sequentially and/or simultaneously. One or more linear actuators (e.g., on cassette 9357 or on a driver) can be configured to actuate the bellows 9303a,b. Alternatively, the devices 9300, 9310 can be rigidized and de-rigidized through one or more sumps or pressure sources (e.g., via a pressure line 9305z). Other mechanisms causing rigidization of the inner and outer rigidizing devices 9310, 9300 are also possible. For example, in some examples, cassette 9357 can include a syringe or other container comprising a fluid that can be delivered to the inner and outer rigidizing devices 9310, 9300 to add pressure for rigidization. In some examples, a syringe or other container can be used to draw fluid within the cassette 9357, creating a vacuum that can be applied to the inner and outer rigidizing devices 9310, 9300.

The cassette 9357 may include a connector 9315y for connecting to additional lumens and/or wiring in the inner rigidizing device 9310. The connector 9315y may include a connection for the delivery of both suction and water to the tip of the inner rigidizing device 9310. The connector 9315y may include electrical connector to connect to a camera mounted to the tip of inner rigidizing device 9310 to an external monitor and/or video processing unit. The connector 9315y may include a mechanical connector that connects to a hollow tube (e.g., working channel) leading all the way to the tip of the inner rigidizing device 9310. By including the connector 9315y, the control of all components of the system 9300z can be performed with the cassette 9357.

Disks 9389, 9371a, 9371b and cams 9374a, 9374b (or the corresponding bellows)

may be accessible from the bottom of the cassette 9357. Disks 9389, 9371a, 9371b and/or cams 9374a, 9374b may have features, such as splines, pins or teeth, to transmit torque. These features can allow the disks 9389, 9371a, 9371b and/or cams 9374a, 9374b to be manipulated (e.g., by a drive unit/robotic driver).

In any of the apparatuses described herein the variable stiffness layer may comprise a plurality of strand lengths that cross each other, including crossing over each other, and that may be compressed to rigidize. As used herein the variable stiffness layer may be formed of one or more strands forming the plurality of strand lengths, which may be knitted, woven, braided, etc. FIGS. 1D to 1G illustrate examples of knitted, woven and braided materials, respectively.

For example, FIGS. 1D-1E illustrate two different knits 600, 600′ that may be used to form the rigidizing layer, also referred to herein as the variable stiffness layer. FIG. 1D shows a weft knit. In this example the knit is formed of one or more strand lengths (which may be a continuous strand of a plurality of separate strands, including broken/cut strands), forming stitch loops 602 that each include a head region 604, a pair of legs 606 and a first and second foot 608 where each foot engages with the head of a stitch loop in a course above or below the original stitch loop course. The connection between the feet of adjacent stitch loops may be referred to as the sinker (the sinker may also correspond to a head when the knit is rotated 180 degrees). In FIG. 1D the wale direct 612 extends up/down, and the course 610 extends right to left. Typically, a wale is a column of loops running lengthwise, corresponding to the warp of woven fabric in FIG. 1D. The course is a crosswise row of loops, corresponding to the filling of the resulting knit.

FIG. 1E illustrates an example of a warp knit 600′. In this example the warp knit also has a course 610′ and wale 612′ direction but the feet of each loop engage with the head region of a knit loop in a row (in the course direction) that is offset, as shown, forming a pattern of overlap 612 and underlap 614 lengths. The knit variable stiffness layers (rigidizing layers) described herein may use any appropriate pattern and may arrange the direction (course or wale direction) relative to the elongate axis (length) of the device. For example, the knit structure (the knit variable stiffness layer) may be configured so that a wale direction of the knit extends in a long axis of the flexible tube. Alternatively, the knit structure may be configured so that a wale direction of the knit structure is perpendicular to a long axis of the flexible tube. Depending on the stitch length (y) relative to the loop diameter (p) and/or the spacing between loops (n), which may be related, it may be beneficial to arrange the knit variable stiffness layers (rigidizing layers) so that that either the wale or the course is arranged in parallel or perpendicular to the long axis of the elongate body of the rigidizable device. In any of the examples described herein, the knit structure may comprise an average loop length that is longer than the loop width. For example, the loop length may be two times or greater (e.g., 3×, 4×, 5×, 6×, 7×, 8×, 9×, 10×, 20×, 40×, 60×, 80×, 100× or more) than an average loop width. Because knits (including knit tubes) may be stretched and compressed in bending without buckling or wrinkling, they may be particularly useful in the rigidizable devices described herein.

Any of the rigidizable devices described herein (and any nested systems or methods including them) may include a rigidizing layer that is woven. For example, a weave may include a plurality of parallel fiber lengths that form a set of intersecting fiber lengths. The fibers may intersect with each other at 90 degree angles, but this angle may vary (e.g., between about 30 degrees and 150 degrees, 45 degrees and 135 degrees, 50 degrees and 130 degrees, 70 degrees and 110 degrees, 80 degrees and 100 degrees, etc.). The pattern of intersecting filament lengths (e.g., the array of filament lengths) may include individual filament lengths that cross over and under each other. For example a pattern may include an under-over pattern, a one over, one under pattern, a two over two under pattern, a two over and one under pattern, etc. Any appropriate fiber (e.g., strand) may be used to form the rigidizing layer, a mentioned for knit rigidizing layers above. For example, the fiber may be a muti-filament fiber including a bundle of multiple filaments forming each strand. The woven pattern may be any desired tightness (e.g., pore size). In general, multiple different lengths of fibers may be used to form the woven pattern.

FIGS. 1F and 1G illustrate examples of braided rigidizing layers. In FIG. 1F, the braid 650 is formed of a plurality of fibers 668, 678 that are arranged in an over-and-under pattern having a braid angle relative to the long axis (e.g., the long axis of the device when included as the rigidizing layer). In general, the braid angle (relative to the centerline along the central axis) of the braided rigidizing layer (tube) may be 45 degrees or less (e.g. less than 45 degrees, 40 degrees or less, less than 40 degrees, 35 degrees or less, less than 35 degrees, 30 degrees or less, 20 degrees or less, less than 20 degrees, etc. In FIG. IF the different filaments forming the braid layer are continuous and unbroken. However in some examples it may be beneficial to include breaks or cuts, as illustrated in FIG. 1G. In this example, the material includes a plurality of breaks or cuts 688 in the braided strands. Although such an arrangement may be undesirable in a fabric or even in a braid used as part of a medical device, this disrupted (e.g., broken or cut) arrangement may be beneficial in the context of a rigidizing layer. Thus, in

FIG. 1G the braided pattern 650′ forming the rigidizing layer (e.g., rigidizing tube) may enhance flexibility in the un-rigidized configuration, while permitting a high degree of rigidizing in the actuated state. Thus, in FIG. 1G the strands 668, 678′ cross over and under each other in the braid pattern shown but are cut 688 periodically along their lengths. The number or density of the cuts may be varied; in some examples the fibers may be cut after every crossing over or under another fiber, while in other examples the fibers may be cut after every 2 (or 3, or 4, or 5, or more) crossings. The cut pattern may be non-uniform. In some examples it may be beneficial to have the cuts or breaks distributed at a density of between about one cut/break for every third crossing, etc. (e.g., between every second and every 25th crossing, every third and every 20th crossing, etc.).

Any of the rigidizable devices described herein (and any system or methods including them) may include a variable stiffness layer (e.g., a rigidizing layer) that is woven. FIG. 1H illustrates an example of a woven a rigidizing layer 705 that may be used as the variable stiffness layer of the rigidizable device. In FIG. 1H the weave includes a plurality of parallel fibers or filaments 718, 728 that form a set of intersecting lengths; in FIG. 1H, the fibers intersect with each other at about 90 degree angles, but this angle may vary (e.g., between about 30 degrees and 150 degrees, 45 degrees and 135 degrees, 50 degrees and 130 degrees, 70 degrees and 110 degrees, 80 degrees and 100 degrees, etc.). The pattern of intersecting filament lengths (e.g., the array of filament lengths) includes individual filament lengths that cross over and under each other as shown; a first filament length 718 crosses over a second filament length 728 and under a third filament length. In this example, the pattern shown in FIG. 1H is an under-over pattern, but this pattern may be different for other examples of rigidizing layers. In some examples the pattern may be two over two under, or two over and one under, etc. Any appropriate fiber (e.g., filament, strand, etc.) may be used to form the variable stiffness layer. The fiber may be a mutli-filament fiber including a bundle of multiple filaments forming each fiber. In some examples the fiber (strand, filament, etc.) is a monofilament. The woven pattern may be any desired tightness (e.g., pore size).

Other rigidizing layers (e.g., knit, woven, etc.) may also include breaks or cuts. These breaks or cuts may be formed during fabrication by laser cutting, mechanical cutting, or any other appropriate cutting technique.

A rigidizing apparatus, once rigidized, may lock into the shape it was in before vacuum or pressure was applied, and the rigidizing process may be performed without changing the shape of the apparatus during the transition from flexible to rigid, i.e., it does not straighten, bend, or otherwise substantially modify its shape (e.g., it may stiffen in a looped configuration, a serpentine shape, a curve, etc.). Upon release of the vacuum or pressure, braids or strands within the layers forming the device can unlock relative to one another and again move so as to allow lower force bending (i.e., enhanced flexibility) of the rigidizing device. As the rigidizing device is made more flexible through the release of vacuum or pressure it may continue to maintain the shape it was in before the vacuum or pressure was released, i.e., it does not straighten, bend, or otherwise substantially modify its shape. Thus, the rigidizing devices described herein can transition from a flexible, less-stiff configuration to a rigid configuration of higher stiffness by restricting the motion between the strands of braid (e.g., by applying vacuum or pressure).

In some examples, the rigidizing apparatuses described herein are configured to toggle between a rigid configuration and a flexible configuration quickly, and with an indefinite number of transition cycles. In some examples the degree of rigidization (e.g., the stiffness) of the apparatus may also be adjusted, for example, by adjusting the positive pressure (in examples that are rigidized by positive pressure) or vacuum (in examples rigidized by vacuum). As interventional medical devices are made longer and inserted deeper into the human body, and as they are expected to do more exacting therapeutic procedures, there is an increased need for precision and control. Selectively rigidizing devices (including selectively rigidizing overtubes) as described herein can advantageously provide both the benefits of flexibility (when needed) and the benefits of stiffness (when needed). Further, the rigidizing devices described herein can be used, for example, with classic endoscopes, colonoscopes, robotic systems, and/or navigation systems, such as those described in International Patent Application No. PCT/US2016/050290, filed Sep. 2, 2016, titled “DEVICE FOR ENDOSCOPIC ADVANCEMENT THROUGH THE SMALL INTESTINE,” the entirety of which is incorporated by referenced herein.

The rigidizing apparatuses described herein can additionally or alternatively include any of the features described with respect to International Patent Application No. PCT/US2016/050290, filed on Sep. 2, 2016, titled “DEVICE FOR ENDOSCOPIC ADVANCEMENT THROUGH THE SMALL INTESTINE,” published as WO 2017/041052, International Patent Application No. PCT/US2018/042946, filed on Jul. 19, 2018, titled “DYNAMICALLY RIGIDIZING OVERTUBE,” published as WO 2019/018682, International Patent Application No. PCT/US2019/042650, filed on Jul. 19, 2019, titled “DYNAMICALLY RIGIDIZING COMPOSITE MEDICAL STRUCTURES,” published as WO 2020/018934, and International Patent Application No. PCT/US2020/013937 filed on Jan. 16, 2020, titled “DYNAMICALLY RIGIDIZING COMPOSITE MEDICAL STRUCTURES,” the entireties of which are incorporated by reference herein.

The rigidizing apparatuses described herein can be provided in multiple configurations, including different lengths and diameters. In some examples, the rigidizing devices can include working channels (for instance, for allowing the passage of typical endoscopic tools within the body of the rigidizing device), balloons, nested elements, and/or side-loading features.

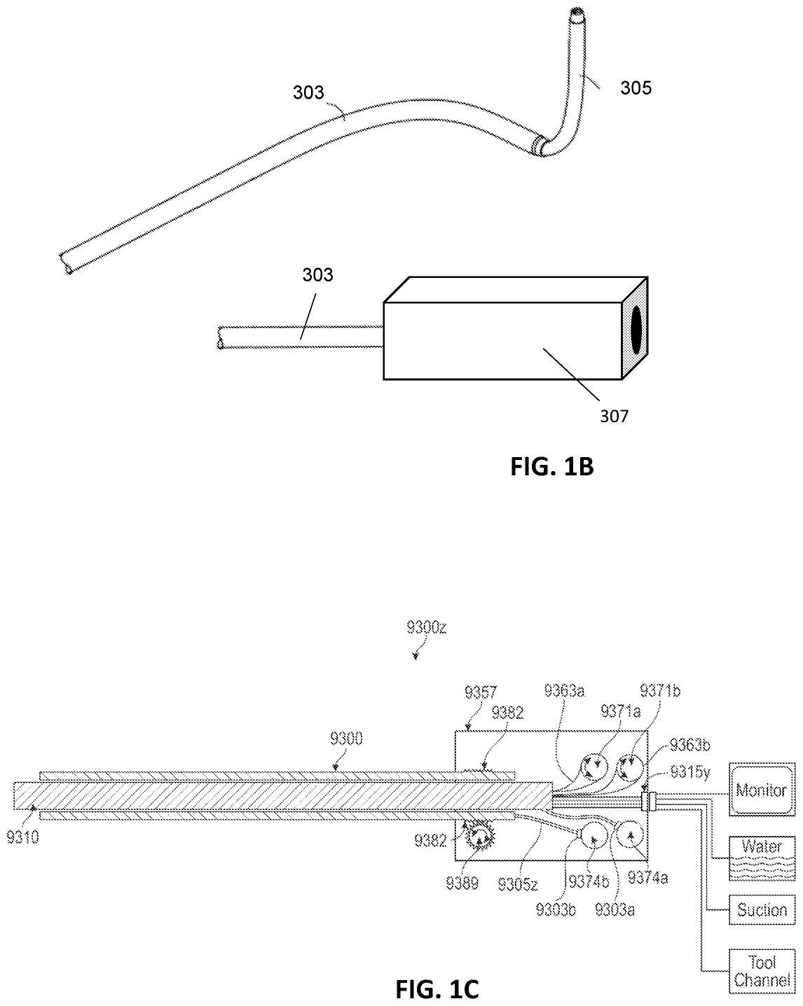

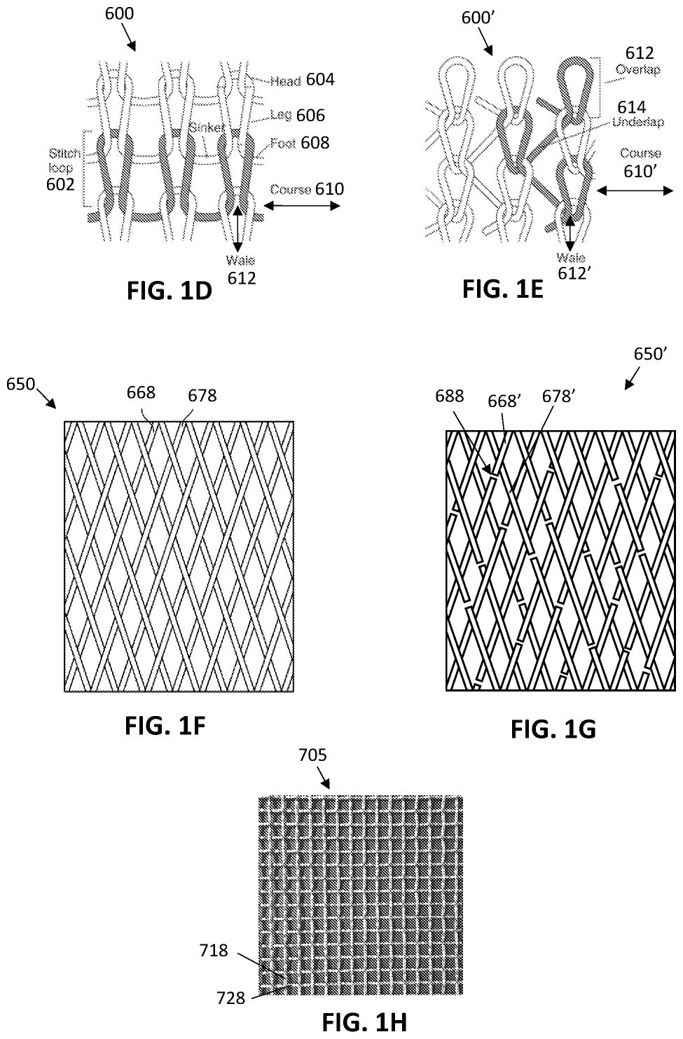

For example, a rigidizing apparatus 100 (also referred to as an apparatus, e.g., system and/or device, including a rigidizable member) may be configured to be rigidized by the application of vacuum, e.g., negative pressure. These apparatuses may generally be formed of layers that are configured to form a laminates structure when negative pressure is applied, so that one or more braided or woven layers may be reversibly fused to a flexible outer layer that is driven against a more radially stiff inner layer. FIGS. 2A-2B illustrate one example of a section through a rigidizing member of an apparatus (e.g., device, system) that is rigidized by the application of vacuum. FIG. 2B shows an enlarged view of the arrangement of the layers of FIG. 2A in the un-rigidized configuration. In this example, the rigidizable member includes an innermost layer 115 that is configured to provide an inner surface against which the remaining layers can be consolidated (e.g., when vacuum is applied). The innermost layer 115 can include a reinforcement element or coil. In particular, the innermost layer 115 may be configured as an ICWT including the ICWTs described herein, e.g., formed of an outer wound wire (e.g., cable, ribbon, wire, etc.) that is continuous with an inner wound wire arranged on both sides of a layer or tube (e.g., a tie layer). The rigidizing member may also include a slip layer 113 over (e.g., radially outwards of) the innermost layer. The slip layer may be, e.g., a lubrication, coating and/or powder (e.g., talcum powder) on the outer surface of the inner layer 115 and/or within the gap layer 111. A radial gap layer 111 may separate the slip layer 113 from a braid or woven layer 109 (referred to herein for convenience as a “braid layer”), providing a space between the braid layer and the slip layer for the braided layer(s) thereover to move within, e.g., when no vacuum is applied; this space or gap may be removed when vacuum is applied, allowing the braided or woven layer(s) to move radially inward upon application of vacuum. A second gap layer 107 may be present between the braid layer 109 and may be similar to layer 111. As will be described in reference to FIGS. 3C-3F, multiple braid layers may be included (e.g., 2, 3 4 or more braid layers may be included) and may be separated by additional gap layers and/or slip layers. The outermost layer 101 may be separated from the braid layer(s) by a gap layer and may be configured to move radially inward when a vacuum is applied to pull down against the braid layer(s) and conform onto the surface(s) thereof. The outermost layer 101 can be soft and atraumatic and can be sealed at both ends to create a vacuum-tight chamber with the innermost layer 115. The outermost layer 101 can be elastomeric, e.g., made of urethane. The hardness of the outermost layer 101 can be, for example, 30A to 80A. Further, the outermost layer 101 can have a thickness of 0.0001-0.01″, such as approximately 0.001″, 0.002, 0.003″ or 0.004″. Alternatively, the outermost layer can be plastic, including, for example, LDPE, nylon, or PEEK.

Any of these apparatuses may include multiple braid layers; the apparatus may include a tube having a wall formed of a plurality of layers positioned around a lumen 120 (e.g., for placement of an instrument or endoscope therethrough). A vacuum can be supplied between the layers to rigidize the rigidizing device 100. Any of the tubular apparatuses described herein may instead include a solid core forming the inner layer 115.

The innermost layer 115 can be configured to provide an inner surface against which the remaining layers can be consolidated, for example, when a vacuum is applied within the walls of the rigidizing device 100. The structure can be configured to minimize bend force and/or maximize flexibility in the non-vacuum condition. As mentioned, the innermost layer 115 may be an ICWT that can include a reinforcement element 150z or coil within a matrix (e.g., in some example, a tie layer), as described in greater detail below.

The layer 109 can be a first braid layer including braided strands 133 similar to as described elsewhere herein. The braid layer can be, for example, 0.001″ to 0.040″ thick. For example, a braid layer can be 0.001″, 0.003″, 0.005″, 0.010″, 0.015″, 0.020″, 0.025″ or 0.030″ thick. In some examples the braid can have tensile or hoop fibers 137. Hoop fibers 137 can be spiraled and/or woven into a braid layer. Further, the hoop fibers 137 can be positioned at 2-50, e.g., 20-40 hoops per inch. In some examples, the rigidizing devices described herein can have more than one braid layer. For example, the rigidizing devices can include two, three, or four braid layers.

In some examples, the outermost layer 101 can include a lubrication, coating and/or powder (e.g., talcum powder) on the outer surface thereof to improve sliding of the rigidizing device through the anatomy. The coating can be hydrophilic (e.g., a Hydromer® coating or a Surmodics® coating) or hydrophobic (e.g., a fluoropolymer). The coating can be applied, for example, by dipping, swabbing, painting, or spraying the coating thereon. The innermost layer 115 can similarly include a lubrication, coating (e.g., hydrophilic or hydrophobic coating), and/or powder (e.g., talcum powder) on the inner surface thereof configured to allow the bordering layers to more easily shear relative to each other, particularly when no vacuum is applied to the rigidizing device 100, to maximize flexibility.

A vacuum can be carried within rigidizing device 100 from minimal to full atmospheric vacuum (e.g., approximately 14.7 psi). In some examples, there can be a bleed valve, regulator, or pump control such that vacuum is bled down to any intermediate level to provide a variable stiffness capability. The vacuum pressure can advantageously be used to rigidize the rigidizing device structure by compressing the layer(s) of braided sleeve against neighboring layers. Braid is naturally flexible in bending (i.e. when bent normal to its longitudinal axis), and the lattice structure formed by the interlaced strands distort as the sleeve is bent in order for the braid to conform to the bent shape while resting on the inner layers. This results in lattice geometries where the corner angles of each lattice element change as the braided sleeve bends. When compressed between conformal materials, such as the layers described herein, the lattice elements become locked at their current angles and have enhanced capability to resist deformation upon application of vacuum, thereby rigidizing the entire structure in bending when vacuum is applied. Further, in some examples, the hoop fibers through or over the braid can carry tensile loads that help to prevent local buckling of the braid at high applied bending load. The stiffness of the rigidizing device 100 can increase from 2-fold to over 30-fold, for instance 10-fold, 15-fold, or 20-fold, or 50-fold, or 100-fold, when transitioned from the flexible configuration to the rigid configuration. In some examples of a vacuum rigidizing device 100, there can be only one braid layer. In other examples of a vacuum rigidizing device 100, there can be two, three, or more braid layers. In some examples, one or more of the radial gap layers or slip layers of rigidizing device 100 can be removed. In some examples, some or all of the slip layers of the rigidizing device 100 can be removed.

The braid layers described herein can act as a variable stiffness layer. The variable stiffness layer can include one or more variable stiffness elements or structures that, when activated (e.g., when vacuum is applied), the bending stiffness and/or shear resistance is increased, resulting in higher rigidity. Other variable stiffness elements can be used in addition to or in place of the braid layer. In some examples, engagers can be used as a variable stiffness element, as described in International Patent Application No. PCT/US2018/042946, filed Jul. 19, 2018, titled “DYNAMICALLY RIGIDIZING OVERTUBE,” the entirety of which is incorporated by reference herein. Alternatively or additionally, the variable stiffness element can include particles or granules, jamming layers, scales, rigidizing axial members, rigidizers, longitudinal members or substantially longitudinal members.

The rigidizable apparatuses described herein may also be rigidized by the application of positive pressure, rather than vacuum. For example, referring to FIGS. 3A-3B, the rigidizing apparatus (e.g., device or system) 2100 can be similar to rigidizing apparatus 100 described above, except that it can be configured to hold pressure (e.g., of greater than 1 atm) therein for rigidization rather than vacuum. A pressure-activated rigidizing device 2100 can also include a plurality of layers positioned around a lumen 2120 (e.g., for placement of an instrument or endoscope therethrough).

For example, FIGS. 3A-3B illustrate longitudinal and radial sections through an example of a pressure-activated rigidizable member of a rigidizing apparatus. The rigidizing device 2100 shown in FIGS. 3A and 3B can include an innermost layer 2115 (similar to innermost layer 115), which may be an inner coil-wound tube, as described above and in greater detail below. The rigidizing device 2100 may also include a slip layer 2113 (similar to slip layer 113), a pressure gap 2112, a bladder layer 2121, a gap layer 2111 (similar to gap layer 111), a braid layer 2109 (similar to braid layer 109) or other variable stiffness layer as described herein, a gap layer 2107 (similar to layer 107), and an outermost containment layer 2101. In any of these apparatuses, the gap regions (e.g., pressure gap, gap layers, etc.) may have variable gap distances or may have a constant gap distance. For example, an apparatus having a tapered OCWT diameter may have a tapered gap such that one end of the device may have a bigger gap forming the gap region than the other end of the device. The end with the larger gap may be more flexible in the flexible state than the end with a smaller gap. This may provide a variable stiffness along the length of the device in the flexible state. Variable stiffness catheters may be advantageous for trackability and using a variable gap distance (which does not affect the rigid state) may be an efficient and effective way to achieve variable stiffness in the flexible state.

The pressure gap 2112 can be a sealed chamber that provides a gap for the application of pressure to layers of rigidizing device 2100. The pressure can be supplied to the pressure gap 2112 using a fluid or gas inflation/pressure media. The inflation/pressure media can be water or saline or, for example, a lubricating fluid such as oil or glycerin. The lubricating fluid can, for example, help the layers of the rigidizing device 2100 flow over one another in the flexible configuration. The inflation/pressure media can be supplied to the gap 2112 during rigidization of the rigidizing device 2100 and can be partially or fully evacuated therefrom to transform the rigidizing device 2100 back to the flexible configuration. In some examples, the pressure gap 2112 of the rigidizing device 2100 can be connected to a pre-filled pressure source, such as a pre-filled syringe or a pre-filled insufflator, thereby reducing the physician's required set-up time.

The bladder layer (or “bladder”) 2121 can be made, for example, of a low durometer elastomer (e.g., of shore 20A to 70A) or a thin plastic sheet. The bladder layer 2121 can be formed out of a thin sheet of plastic or rubber that has been sealed lengthwise to form a tube. The lengthwise seal can be, for instance, a butt or lap joint. For instance, a lap joint can be formed in a lengthwise fashion in a sheet of rubber by melting the rubber at the lap joint or by using an adhesive. In some examples, the bladder layer 2121 can be 0.0002-0.020″ thick, such as approximately 0.005″ thick. The bladder layer 2121 can be soft, high-friction, stretchy, and/or able to wrinkle easily. In some examples, the bladder layer 2121 is a polyolefin or a PET. The bladder 2121 can be formed, for example, by using methods used to form heat shrink tubing, such as extrusion of a base material and then wall thinning with heat, pressure and/or radiation. When pressure is supplied through the pressure gap 2112, the bladder layer 2121 can expand through the gap layer 2111 to push the braid layer 2109 against the outermost containment layer 2101 such that the relative motion of the braid strands is reduced. Any of the apparatuses descried herein may include multiple filling locations for the bladder and/or a distally located filling location for the bladder layer. Any of the apparatuses described herein may include a bladder that is configured to prevent sticking, e.g., sticking in the closed configuration, as described in greater detail below.

The outermost containment layer 2101 can be a tube, such as an extruded tube. Alternatively, the outermost containment layer 2101 can be a tube in which a reinforcing member (for example, metal wire, including round or rectangular cross-sections) is encapsulated within an elastomeric matrix, similar to as described with respect to the innermost layer for other examples described herein. In some examples, the outermost containment layer 2101 can include a helical spring (e.g., made of circular or flat wire), and/or a tubular braid (such as one made from round or flat metal wire) and a thin elastomeric sheet that is not bonded to the other elements in the layer. The outermost containment layer 2101 can be a tubular structure with a continuous and smooth surface. This can facilitate an outer member that slides against it in close proximity and with locally high contact loads (e.g., a nested configuration as described further herein). Further, the outer layer 2101 can be configured to support compressive loads, such as pinching. Additionally, the outer layer 2101 (e.g., with a reinforcement element therein) can be configured to prevent the rigidizing device 2100 from changing diameter even when pressure is applied.

Because both the outer layer 2101 and the inner layer 2115 (e.g., ICWT) may include reinforcement elements therein, the braid layer 2109 can be reasonably constrained from both shrinking diameter (under tensile loads) and growing in diameter (under compression loads).

By using pressure rather than vacuum to transition from the flexible state to the rigid state, the rigidity of the rigidizing device 2100 can be increased. For example, in some examples, the pressure supplied to the pressure gap 2112 can be between 1 and 40 atmospheres, such as between 2 and 40 atmospheres, such as between 4 and 20 atmospheres, such as between 5 and 10 atmospheres. In some examples, the pressure supplied is approximate 2 atm, approximately 4 atmospheres, approximately 5 atmospheres, approximately 10 atmospheres, approximately 20 atmospheres. In some examples, the rigidizing device 2100 can exhibit change in relative bending stiffness (as measured in a simple cantilevered configuration) from the flexible configuration to the rigid configuration of 2-100 times, such as 10-80 times, such as 20-50 times. For example, the rigidizing device 2100 can have a change in relative bending stiffness from the flexible configuration to the rigid configuration of approximately 10, 15, 20, or 25, 30, 40, 50, or over 100 times.

### Inner Coil Wound Tube (ICWT)

In general, described herein are rigidizing apparatuses that may include an inner coil wound tube (ICWT) as the innermost layer 115, which can include a reinforcement element such as a coil or coils. The ICWT may be configured to be highly flexible, so that it may bend easily, e.g., with very low force, while providing a large diameter opening (lumen) and having a very high compressive strength, preventing it from collapsing under externally applied (e.g., inward) pressure. The inner diameter of the lumen may be 2 mm or greater, 3 mm or greater, 4 mm or greater, 5 mm or greater, 6 mm or greater, 7 mm or greater, 8 mm or greater, 9 mm or greater, 10 mm or greater, 11 mm or greater, 12 mm or greater, 14 mm or greater, 15 mm or greater, 20 mm or greater, 25 mm or greater, 30 mm or greater, 35 mm or greater, 40 mm or greater, 45 mm or greater, 50 mm or greater, 55 mm or greater, 60 mm or greater, 65 mm or greater, 70 mm or greater, 75 mm or greater, etc.

In general, the ICWTs described herein are thin-walled structures, which may decrease both the weight and increase the flexibility of the ICWT. For example, the wall thickness may be about 2 mm or less, about 1.75 mm or less, about 1.5 mm or less, about 1.25 mm or less, about 1.0 mm or less about 0.9 mm or less, about 0.8 mm or less, about 0.7 mm or less, about 0.6 mm or less, about 0.5 mm or less, about.25 mm or less, etc.

An ICWT may be a wire-reinforced tube (or may be formed from a tubular extrusions or from sheets or strips of material that is or are spiral wrapped to create a tube that is wire reinforced). The wire-reinforced tube may include a matrix, which may be an inner layer or tube formed of a polymeric material. The matrix may be positioned between an inner and an outer wire. The term “wire” in the context of the ICWT may refer to any elongate support member such as a cable, ribbon, filament, etc. that may be wound both internally and externally of the tubular ICWT. In some examples the wire of the ICWT is a substantially flat, ribbon-shaped wire having a width that is greater than the thickness, and at least two sides that are substantially parallel. The edges may be squared-off, rounded, beveled, etc.

Thus, an ICWT may include an intermediate matrix that is highly flexible that is wrapped externally by coils (e.g., a helically wound coil) of wire (e.g., ribbon). The inner and/or outer surface of the ICWT may also be covered and/or laminated with a flexible polymeric material. In some cases this flexible polymeric covering material may be an elastomeric material that is hydrolytically stable, so that it does not soften or change upon exposure to fluid, e.g., water. Any of these ICWTs may include one or more coatings on the inner and/or outer surfaces of the ICWT. For example, the ICWT may include a lubricious coating or material (e.g., the flexible polymeric material) may be formed of a material that is lubricious and/or hydrophobic and/or fluid resistant material or that is able to be coated by a lubricious and/or hydrophobic and/or fluid resistant material.

In particular, described herein are apparatuses in which the inner and outer wire coils are formed as a single, continuous wire extending both around the outside of the matrix with a first pitch and around the inside of the matrix with a second pitch. The pitch angle of the outside wire may be the same as the pitch angle of the inside wire, or it may be different.

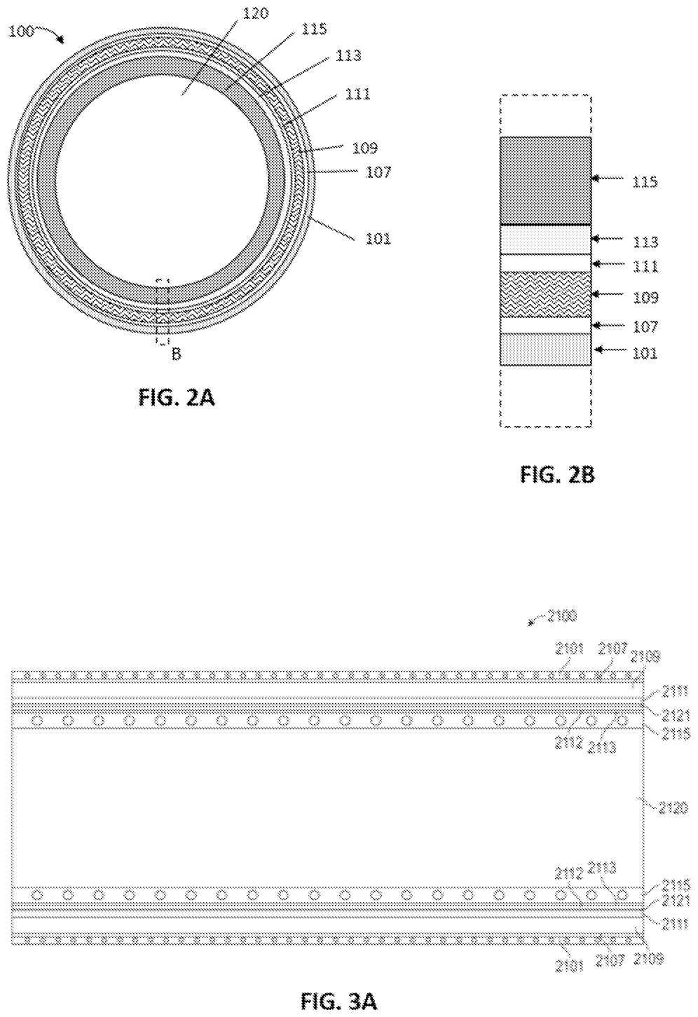

For example, FIG. 4A shows a schematic section through one example of an ICWT that may be used with any of the apparatuses described herein. In this example, the ICWT 415 includes a matrix 465 that is wrapped on the outside by a first coil portion 450 formed by a flat, ribbon-shaped wire, and on the insider by a second coil portion 450′ that is also a flat ribbon-shaped wire. As described in greater detail below, the first coil portion and the second coil portion may be continuous and/or formed of the same wire. The wall of the ICWT may be thin, and may have a thickness 470 that is, e.g., 1 mm or thinner (e.g., 0.5 mm or thinner, 0.3 mm or thinner, 0.1 mm or thinner, etc.). The outer surface may be covered or coated with a polymeric material 467 that may be an elastomeric material and may be bonded (e.g., melted, fused, etc.) over and onto the first coil and matrix. Alternatively or additionally the inner surface may be covered or coated with a polymeric material 467′ that may be the same or a different elastomeric material and may be bonded (e.g., melted, fused, etc.) over the second coil portion and matrix, as shown. The outer 467 and/or inner 467′ polymeric material may be the same material as the intermediate matrix 465 or it may be a different material. In some examples the outer and/or inner polymeric material may be referred to as outer matrix material (in contrast to the intermediate matrix material between the inner 450 and outer 450′ reinforcement members, e.g., coils, wires, etc.).

In FIG. 4A the ICWT dimensions are not shown to scale, as the inner diameter 469 may be much larger relative to the wall thickness 470.

Thus, in any of the ICWTs described herein, the ICWT 415 may include a wire 450 (which may also be referred to more generically a reinforcement element) that wraps around both the inside and the outside of the matrix 465 of the ICWT. In examples of the ICWT in which the inner and outer reinforcement elements (wires) are separate, as shown in FIG. 4B, the distal ends of the reinforcement elements may be loose. However, a loose reinforcement element (wire) may cause significant problems, particularly for medical application, as the loose end(s) may unravel from the device and may also cause harm or damage to the surrounding tissue when inserted into the body, due to protruding sharp points. To reduce these risks, in some examples, it is particularly advantageous to have the reinforcement element (wire) forming the inner and outer coils to be formed of one continuous piece extending between the proximal and distal end of the matrix 465. For example, the reinforcement element 450 can begin at the proximal end of the ICWT, extend to the distal end of the ICWT, then double back over itself, as described in FIG. 4C. This configuration removes wire termination at a distal ends of the ICWT forming the innermost layer of a rigidizing device.

FIG. 4B shows an example of just the reinforcement elements (e.g., wires) forming the inner and outer coils of an ICWT in which the inner and outer coils are distinct wires. In this example the outer wire coil 450 comprise a wire spiraling (helically) on or around a matrix (not shown) and over the inner reinforcement element (e.g., wire) 450′. The reinforcement element 450 of FIG. 4B begins at the proximal end and spirals helically towards the distal end of the

ICWT, where it terminates in a first end 454. Similarly, the inner reinforcement element 450′ spirals within the outer reinforcement layer (on the inner side of the matrix, not shown) to terminate 454′ at the distal end of the ICWT. This configuration may result in loose ends 454, 454′ of the wires at the distal end of the ICWT. These loose ends may potentially stick out, causing manufacturing issues and patient harm. In general, the wire ends may also bias inward (not shown) which would also be problematic, potentially affecting functioning of the rigidizing device, or catching on devices that passed through the center.

FIG. 4C shows an example of a reinforcement element 450 (e.g., wire) of an ICWT in which the inner and outer helically-wound coils reinforcing the apparatus are formed of a single continuous length of wire beginning at the proximal end of the ICWT 415 and extending towards the distal end of the ICWT; at the distal end of the ICWT the reinforcing wire doubles back towards the proximal end of the ICWT. This continuous wind configuration avoids the exposed, e.g., cut, ends of the reinforcing wires at the distal end of the ICWT 415. While a spiral configuration is shown in FIG. 4C, it will be appreciated that other configurations having a continuous structure extending from a proximal portion of the innermost layer (e.g., the proximal end) to the distal end and back to a proximal portion of the innermost layer (e.g., the proximal end) will also result in the absence of wire ends at the distal end of the layer and are contemplated herein. In FIG. 4C the inner and outer lengths of wire forming the inner coil 450′ and outer coil 450 are shown joined 453 (e.g., coupled, fused, etc.). Alternatively, the inner and outer wires may simply be formed of the same wire.

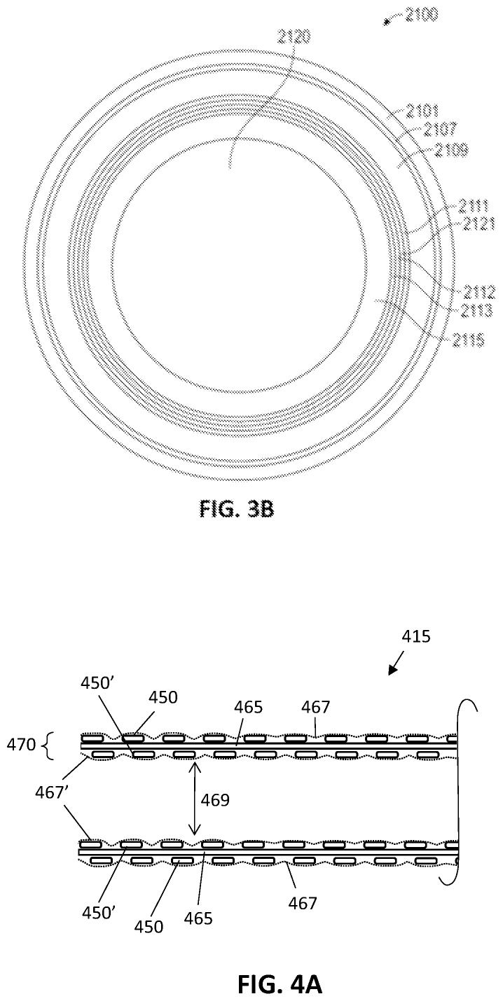

In any of the apparatuses described herein the inner coil region and outer coil region of the reinforcement coil (e.g., wire, ribbon, etc.) may have a uniform pitch or a non-uniform pitch. The pitch of the inner coil region may be the same as the pitch of the outer coil region, or the two may be different. In any of the apparatuses described herein, the inner coil region and outer coil region of the reinforcement element may have a uniform helical angle or a non-uniform helical angle. The helical angle of the inner coil region may be the same as or different from the helical angle of the outer coil region. As used herein the helical angle may refer to the angle between any helix and an axial line through the helically wound coil. For example, FIGS. 5A-5C illustrate examples of inner and outer coil regions of a reinforcement wire or ribbon.

In FIG. 5A, an example of a reinforcement coil having an outer helical coil region 550 and an inner helically-wound coil region 550′. In this example the outer and inner helical coil regions may be formed of the same continuous ribbon that doubles back over itself, as discussed above. An axial line 532 is shown passing through the midline of the apparatus. The inner coils region 550′ and the outer coil region 550 each form a constant helical angle in FIG. 5A, but in opposite directions (e.g., +a and −a). An inner layer 575 is also shown. In some examples a matrix layer (not shown) may be positioned between the inner and outer coil regions.

FIGS. 5A and 5B show the same device but are rotated 90 degrees around long axis (axial line 532). These figures show the helical angles having opposite directions but equivalent degree. Since they are equal to each other, they cross over repeatedly along the length creating a “spine,” which is visible in FIG. 5B, and openings (visible in FIG. 5A). Bending in the direction of the spine is less flexible than the openings. In some examples, it may be beneficial to have equal bending stiffness regardless of orientation. Thus, in some examples it may be beneficial to have helical angles that are different in magnitude, as shown in FIG. 5C. in FIG. 5C, the helical angle (a) of the outer coil region 550 does not equal the helical angle of the inner coil region 550′ (B), thus the crossing and gap region of the inner and outer coil regions do not align along the long axis.

### Bladder Layer

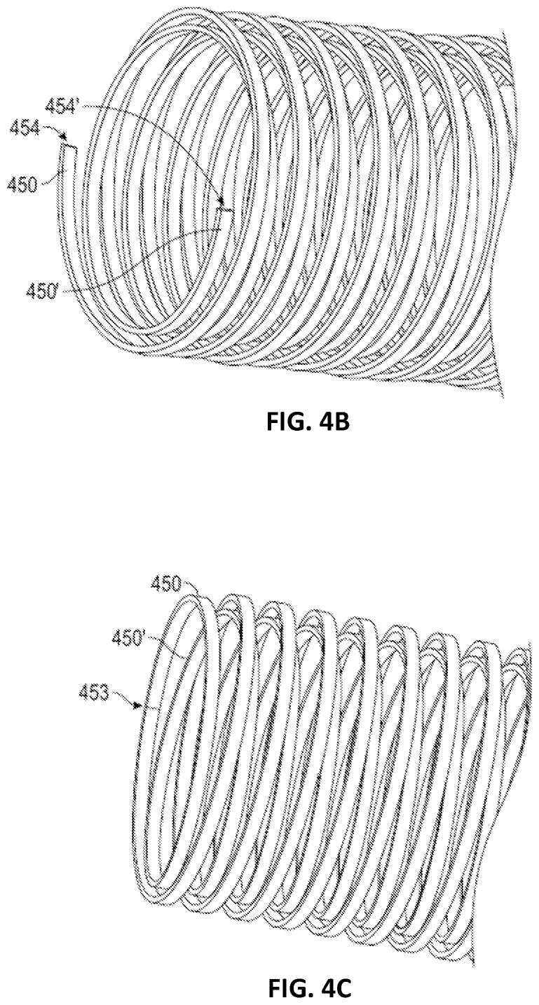

Any of the apparatuses described herein may include a bladder layer that is configured to prevent or reduce sticking of the bladder layer to itself or to an ICWT. For example, FIG. 6 illustrates a section through a rigidizing apparatus (similar to that shown in FIGS. 3A-3B) including a bladder layer 2121 that is adjacent to an ICWT forming the innermost layer 2115 and is separated by a gap 2112 and a slip layer 2113. The bladder layer 2121 may include one or more features configured to prevent the bladder from sticking to itself or the innermost layer 2115. If the bladder layer 2121 does stick to itself or the innermost layer 2115, the bladder may not inflate or may not inflate properly, potentially rendering it non-functional. To prevent this from happening, one or more additives can be added to the bladder layer 2121. For example, slip additives, lubricants, or color additives (e.g., TiO) can be added to the bladder layer 2121.

In some examples, the bladder layer may be textured to prevent sticking. For example, the bladder layer may be ribbed, speckled, may include nubs, etc. This texturing can create space between the opposite layers of the bladder and between the bladder layer and surrounding layers, helping to prevent sticking of the bladder. The spacing may also provide inflation channels from which air (or other inflation material) may be driven.

In some examples, an outer surface of the innermost layer 2115 can be textured (e.g., ribbed, speckled, comprise nubs, etc.). The texturing can be performed by wrapping textured film or a braid around the layer 2115, baking it, and then removing the film to impart the texture onto the innermost layer 2115. In some examples, the texturing is performed by placing the innermost layer 2115 (and/or the bladder layer 2121) into a textured mold and applying heat and/or pressure. Other methods are also contemplated.

In some examples, a braid layer can be added between the innermost layer 2115 and the bladder layer 2121. This layer can separate the innermost layer 2115 and the bladder 2121 to prevent them sticking together. The textured nature of the braid layer can also help prevent it from sticking to surrounding layers.

In some examples, the inner layer 2115 and/or the bladder 2121 can comprise a coating (e.g., parylene) to help prevent sticking.

In some examples, one or more tubes or channels can be positioned between the bladder 2121 and the inner layer 2115 and extend along at least a portion of or their entire length to ensure a fluid pathway exists to inflate the bladder.

### Delamination Prevention or Reduction

In a composite structure, such as the rigidizing tubes described herein, layer adhesion is important. Delamination represents a failed structure and can lead to clinical complications. There is a need for enhanced inter-laminar adhesion. Some of the materials used in the layers of these structures may provide properties appropriate for their function (e.g., within the rigidizing tube), but not very suitable for inter-laminar adhesion. Certain of the layers forming the apparatuses described herein may include one or more tie layers that may create a bondable (e.g., melt bondable or adhesively bonded) surface that improves adhesion to the catheter jacket following the reflow process.

In some examples, a tie layer creates enhanced inter-laminar adhesion. A tie layer may be disposed between layers of the composite tubular structure (e.g., on the other side of a reinforcement). For example, a tie layer may be included as part of an inner coil-wound tube (ICWT) and/or an outer coil-wound tube (OCWT). The bond between the tie layer and surrounding layers (e.g., the reinforcement layer) may prevent the delamination of the layers of the ICWT and/or OCWT. For example, the tie layer may prevent delamination across a reinforcing layer, by requiring a much higher force to pull its adherent partner across the reinforcing element.

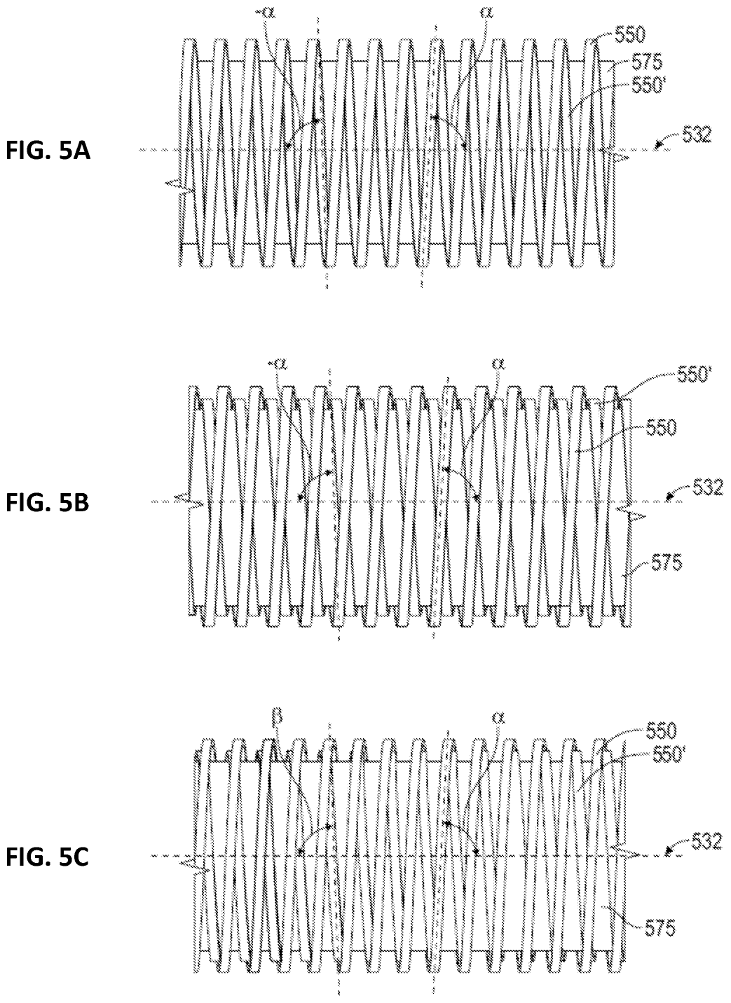

FIG. 7A shows a sectional view through one example of a rigidizing member including an inner coil-wound tub with a tie layer. In FIG. 7A the apparatus includes an inner layer 775, a coil wound layer 750 and a tie layer 776. The inner layer 775 does not bond to the inner coil-wound layer 750, but the tie layer 776 may bond to the intermediate layer 780 (e.g., matrix layer), and the inner layer 775 may bond to the tie layer 776. Similarly, the outer layer 777 may bond to the intermediate layer 780 but not to the outer coli-wound layer 760.

FIGS. 7B and 7C show examples of an outer coil-wound tubular layer (OCWT) of an outer layer 710 and an inner coil-wound tubular layer (ICWT) of an inner layer 715 of composite tubular structures that may be part of a rigidizing device. In general, the ICWT may be structured similar to an OCWT (including using a single metallic coil to form both an inner coil and an outer coil). These examples show a tie layer 774 that may be disposed between the surrounding layers. FIG. 7B shows an inner longitudinal axis 735 of the OCWT. Layer 775 is an inner surface of the inner layer of the OCWT 710. Layer 774 is a tie layer. Layer 750 is the reinforcement element (e.g., a wire coiled around the matrix layer 774). Layer 776 is an outer surface of the OCWT 715. FIG. 7C resembles that shown in FIG. 7B, but for an ICWT. In this example, similar to the ICWT schematic shown in FIG. 4A, the ICWT includes an outer coil 750 and an inner coil 750′ that may be formed of the same wire that doubles back over the distal end of the device (not shown). An outer and/or inner layer 776, 775 may included.

### Methods and Techniques for Modulating Stiffness

In some examples of the apparatuses described herein, the layers of the rigidizing device terminate at different points along the length of the device. For dynamically rigidizing catheters, multiple layers can serve to create tips that are more rigid than desired. Axially separating the distal terminus of the layers can enhance distal tip flexibility. Enhancing distal tip flexibility can enhance distal tip tracking.

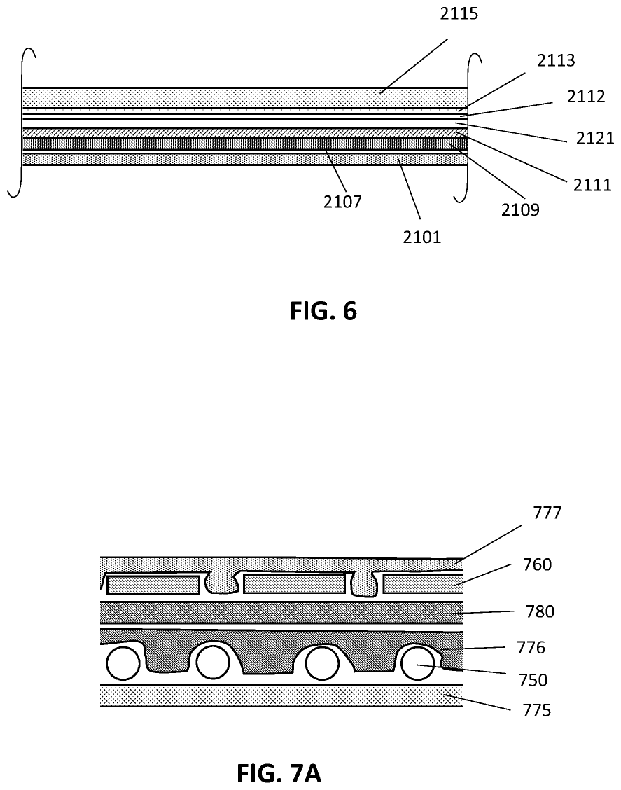

For example, in a rigidizing device comprising an innermost layer 815, a bladder layer 821, a braid layer 809, and an outermost containment layer 801, a distal end of the innermost layer 815 may be separated by a first difference 8102 from the braid layer 809, the bladder layer 821, and the outermost layer 801, as shown in FIGS. 8A and 8B.

In FIG. 8A, the first difference 8102 is larger than in FIG. 8B. As such, the distal tip section 833 is longer in FIG. 8B than in FIG. 8A. This longer first difference 8102 results in a more flexible distal tip for the rigidizing device shown in FIG. 8A.

FIG. 8C shows a plot of stiffness vs. length of the rigidizing device for both the device in FIG. 8A (4A) and the device in FIG. 8B (4B). As shown, the stiffness of the examples diverges where the longer distal tip section 833 begins in FIG. 8A, with that example having a lower distal stiffness region as compared to the example of FIG. 8B.

In some examples, there can be additional differences between the layers. For example, there can be a first difference between the inner layer and the bladder layer distal ends, and a second difference between the bladder layer, braid layer and the outer layer distal ends. In some examples, there can be a first difference between the inner layer and the bladder layer distal ends, a second difference between the bladder layer and the braid layer distal ends, and a third difference between the braid layer and outer layer distal ends. Other permutations and combinations of differences between layer distal ends are also contemplated.

Any of the difference distances can be about 0.1 mm-5 cm, 0.5 mm-4 cm, 1 mm-3 cm, 1 mm-1 cm, etc.

In examples with more than one difference separating distal ends of the layers, the differences can be the same or different.

### Distal End Inflation/Deflation

In some examples, the rigidizing devices disclosed herein comprise a dedicated lumen extending to the distal end that allows the device to inflate/rigidize from the distal end to the proximal end rather than from the proximal end to the distal end. For long length catheters with narrow walls and closely adjacent constituents that might have tendencies to adhere or poorly separate, there can be a risk that inflation is insufficient to provide rigidizing all the way to the distal end.

The dedicated lumen can comprise a lumen underneath or on top of the bladder layer. Other radial locations and axial termination locations are also contemplated.

In some examples, the lumen comprises a thin walled layflat tube. Other lumen configurations (e.g., round tubing) are also possible.

The lumen can comprise a material such as PET. Other materials (e.g., TPU, TPE, PEEK, Mylar, urethane, or silicone) are also possible.

In some examples, the lumen wall comprises a thickness of about 0.0005″, 0.0001-0.001, 0.00001-0.001, etc.

In some examples, the lumen comprises a thin walled layflat tube comprising PET heat shrink tubing that is about 0.0005″ thick.

In some examples, the lumen comprises breather (e.g., breather mechanism) material that serves to prevent face-to-face surface sealing during vacuum inside of it to keep it open during application of vacuum. The device can comprise a one-way valve located in the handle that does not allow application of fluid pressure but does allow application of vacuum. These features may be helpful as the thin heat shrink may collapse and seal on itself during vacuum.

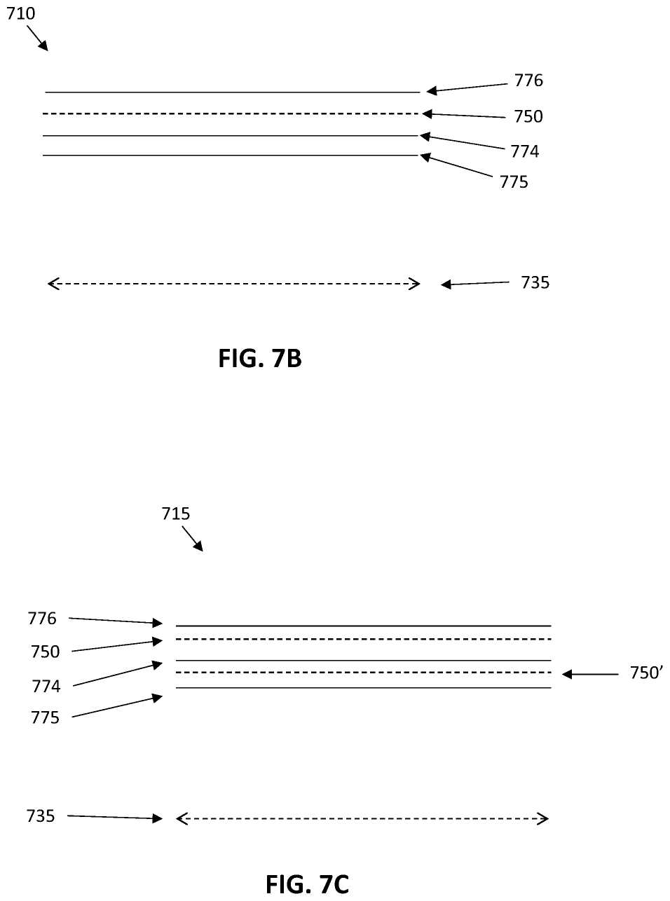

FIG. 9 shows a cross sectional view of an example of a rigidizing device with longitudinal axis 935 comprising a distal end inflation lumen 976x extending between the inner layer 915 and the bladder layer 921. The braid layer 909 is positioned over the bladder layer 921, and the outer layer 901 is positioned over the braid layer. It will be appreciated that this figure does not show any intervening gap or slip layers.

### Stabilized Procedure Base

In some examples, the rigidizing devices described herein can be used to establish a stable and imaginable base from which to perform procedures, especially those procedures performed deep within the body (e.g., in the pulmonary vasculature). In such procedures, it can be a challenge to determine the exact location of the tip of the device. Getting lost is common, which can extend procedure time, delay treatment, may require increased use of contrast and radiation, and may create significant procedural and patient risk.

Using dynamic rigidization enables the creation of ‘Deep Stable’ points that serve as important landmarks for enhanced navigation and treatment. Having the dynamically rigidizing device as a stable reference point from which to navigate and treat is very helpful. Currently available devices, such as a coronary pigtail catheter, would not provide a stable location. When the dynamically rigidizing catheter is advanced and then rigidized, its tip can serve as a fiducial marker for use relative to imagining-for example, angiograms, merged fluoroscopy, CT, DynaCT, ultrasound, etc. Machine learning and Artificial Intelligence could use this fiducial for the guidance of further interventions. In particular, having the entire length of the device rigidized may create a particularly stable distal point.

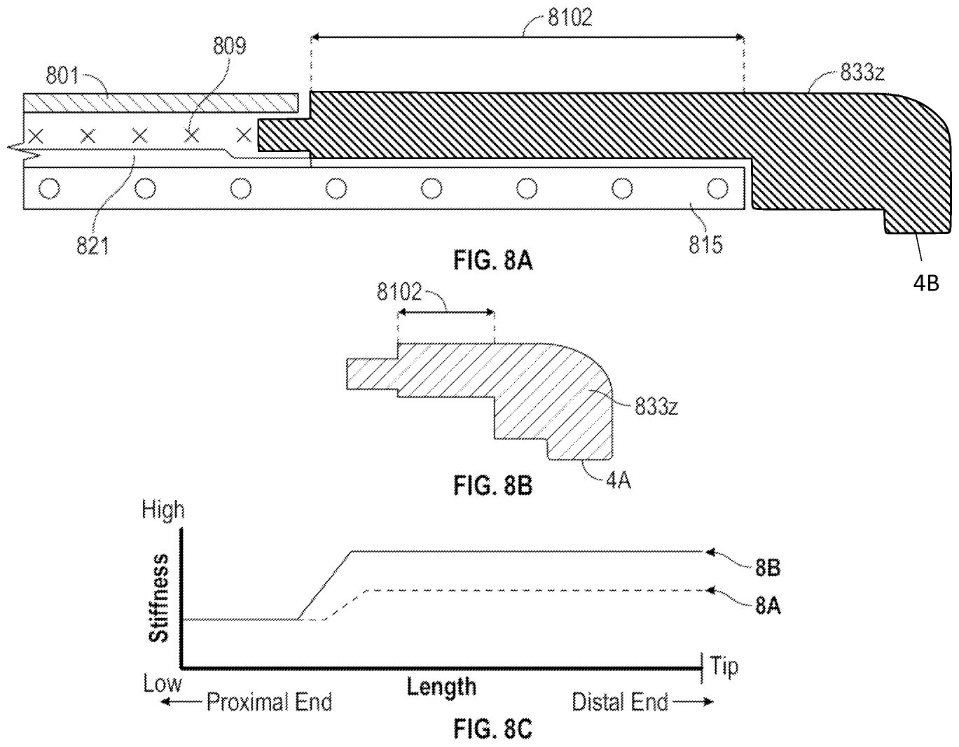

In an exemplary example, as shown in FIG. 10, a dynamically rigidizing catheter 1000 can be navigated through the body (e.g., the vasculature) to a deep treatment site (e.g., pulmonary vasculature) using imaging guidance. At that point the device can be rigidized. A dilator 1077 is within the central bore of the dynamically rigidizing catheter and out of its distal end. When it is withdrawn, a treatment can be performed in the surrounding anatomy-either directly through that lumen, or via the introduction of other devices through that lumen. The distal tip 1055 which can be both stable and readily imaged is shown. Further, the distal tip as a fiducial can be clinically valuable as subsequent procedural elements can be mathematically computed and planned.

### Rigidizing Apparatuses including Particles

In some examples, the dynamically rigidizing catheters described herein comprise particles against which the bladder compresses. Examples of particles may include, e.g., particulate or powders, including but not limited to organic material (e.g., sand (mineral or rock)), plastics, elastomers, or metal.

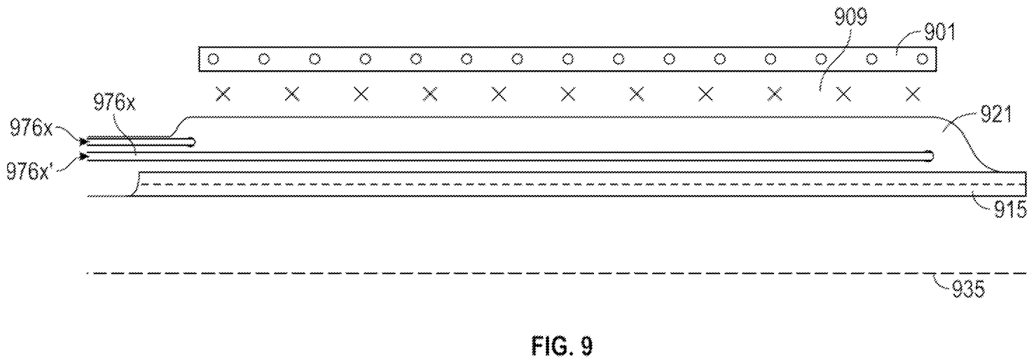

FIG. 11 shows an example of a portion of a catheter in which the particles are compressed along with braid layer 1109. The catheter comprises an inner layer 1115, a bladder layer 1121, a layer with a combination of braid 1109 and particles 1178x, and an outer layer 1101.

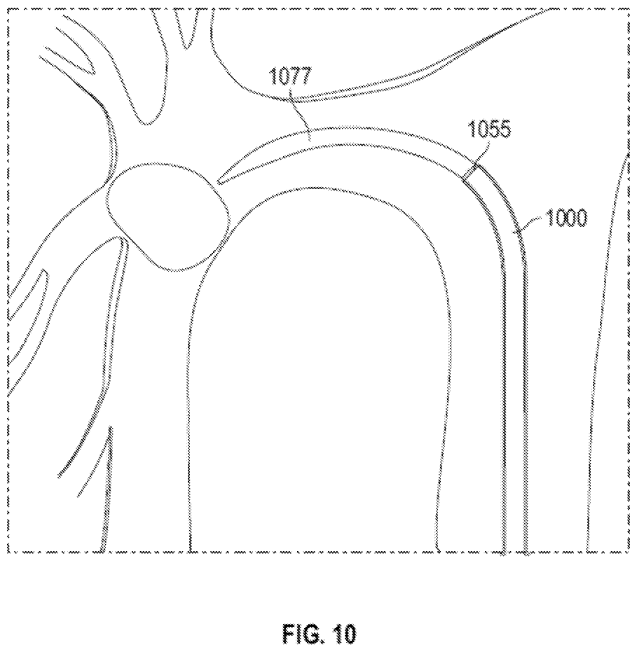

In some examples, as shown in a cross sectional view in FIG. 12, the bladder can compress against the particles alone. The example shown in FIG. 12 comprises an inner layer 1215, a bladder layer 1221, a particle layer 1278, and an outer layer 1201.

It is possible to vary the stiffness along the length of the catheter by varying the use of powder and braid along its length. For instance, it is possible to make a significantly softer tip by eliminating braid from the end of the compressed section, replacing it with powder. Alternatively, if extreme rigidization is required at a specific section, that section can include both braid and powder in compression.

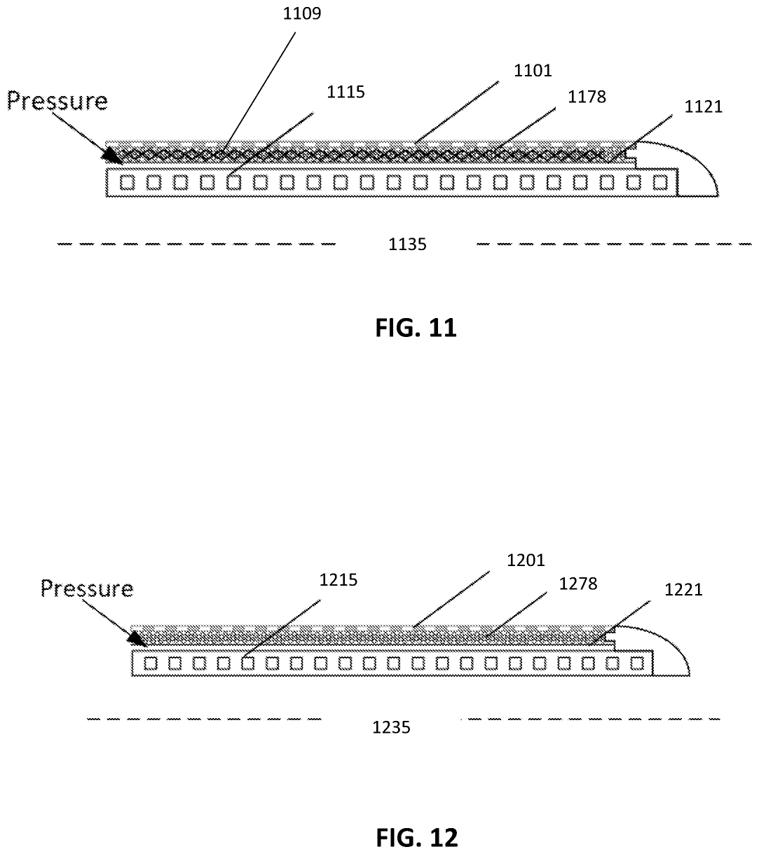

In some examples, a portion of the catheter comprises braid 1309 alone and a portion comprises particles 1378 alone against which the bladder 1321 can compress, as shown in FIG. 13. Also shown in FIG. 13 are the longitudinal axis 1335 of the device and the inner layer 1315 and outer layer 1301.

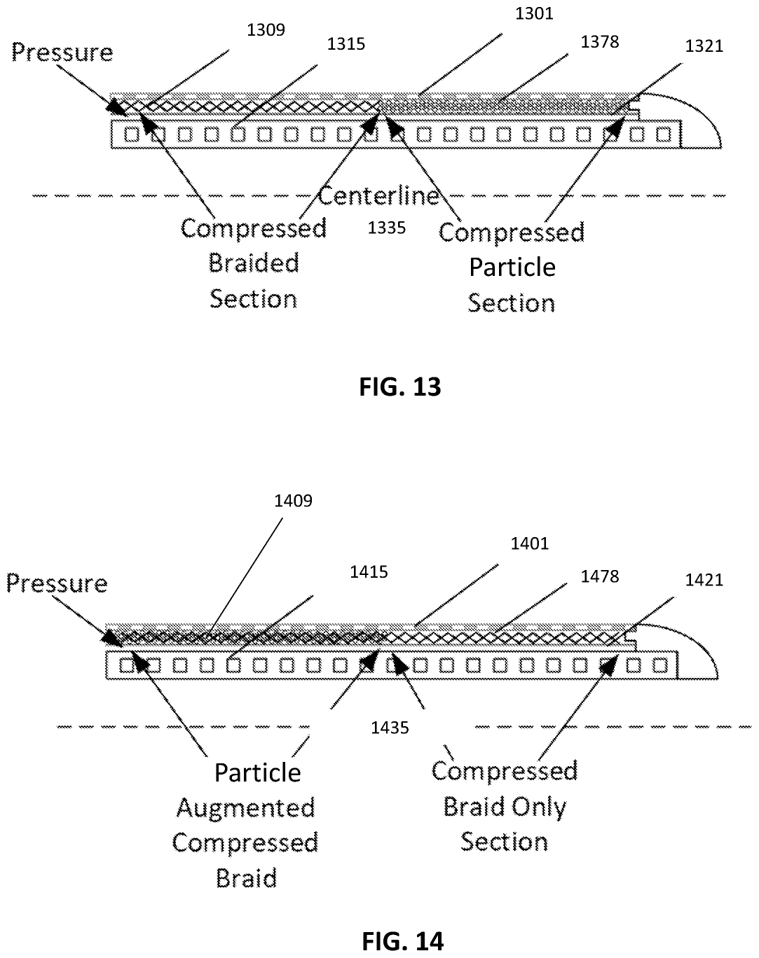

In some examples, a portion of the catheter comprises braid alone 1409, and a portion comprises braid 1409 and particles 1478, as shown in FIG. 14. Also shown in FIG. 14 are the longitudinal axis 1435 of the device and the inner layer 1415 and outer layer 1401.

Other configurations are also possible. For example, in some examples, a portion of the catheter comprises braid alone, a portion comprises particles alone, and a portion comprises a combination of braid and particles against which the bladder can compress.

In some examples, the portions of the catheter comprising particles (either alone or with braid) can comprise a differing amount or composition of particles. Jamming particles can comprise particles, particulate or powders, including but not limited to organic material (e.g., sand (mineral or rock)), plastics, elastomers, or metal. By varying the nature of the powder, e.g., size (1 micron, 10 microns, 100 microns, 1000 microns), shape, porosity, etc., a range of degree of rigidization is possible.

By filling the space the bladder compresses against (whether using vacuum or pressure) with particles in addition to braid, it is possible to make a rigidizing catheter of augmented stiffness.

### Variable Pitch Coil Wound Tubes

The rigidizing apparatuses described herein may be configured to prevent rupture or breakage at the distal and/or proximal ends of the rigidizing apparatus. They may be configured to enhance trackability, and to create an end with a highly engineered stiffness transition profile. As described above, the rigidizing apparatus may include a plurality of layers, including a rigidizing layer (e.g., a variable stiffness layer), a bladder layer that may be driven against the variable stiffness layer in order to increase its stiffness, and therefore increase the rigidity of the apparatus, and one or more reinforced layers, such as an inner coil wound tube (ICWT) and an outer coil wound tube (OCWT). In the non-rigidized state these apparatuses may be highly flexible; in particular it may be advantageous to have the distal end regions of these apparatuses by highly flexible, and to have relatively smooth and compact profiles. Such devices may be particularly beneficial when engaged (e.g., in a nested or telescoping manner) with other devices. The distal ends may be engineered to be non-rigidizing zones, with rigidizing elements (for example, bladder and rigidizing layer) terminating proximally, so as to enhance a gradual increase of stiffness from a non-rigidized distal tip towards a rigidized zone more proximally.

For example, it may be helpful to control the stiffness of the distal end of the rigidizing devices particular in nested systems, so that the transitions across regions of different stiffnesses, including when sliding over each other, may be smooth and continuous. Thus, in any of the apparatuses described herein, the rigidizing devices may include distal end regions that are configured to be more flexible, even when the apparatus is rigidized (or in some cases because that zone is non-rigidizing), than more proximal regions. In some examples, this may be accomplished by including a variable pitch in one or more coil wound tubes (e.g., ICWT, OCWT) within the rigidizing device.

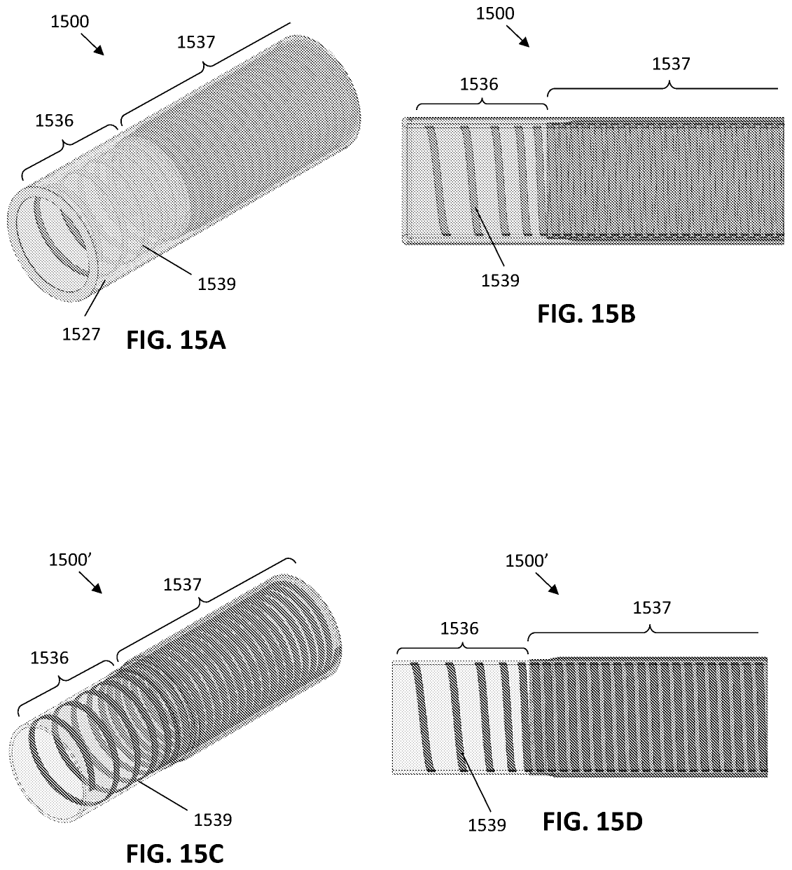

For example, FIGS. 15A-15D illustrate two examples of coil wound tubes that may be included as part of a rigidizing apparatus. In FIG. 15A the distal end region of a coil wound tube 1500 is shown partially transparent. This example includes a proximal region 1537 having both a first, upper, coil that is helically wound around the tube at a first pitch, that is adjacent to a second, lower, coil that may be wound in the opposite direction. Either the upper or the lower coil may extend into the distal end region 1536 of the device, or only one of those reinforcements. Alternatively or additionally the portion of the coil 1539 (upper or lower) that extends distally in the distal region may have an increased or increasing pitch, as shown in FIGS. 15A-15D; in these examples the coil 1539 extending distally in the distal region having a continuously increasing pitch is the lower coil. In FIGS. 15A-15B the example coil wound tube is shown adjacent to or surrounded by a variable stiffness layer (e.g., a knitted layer, a woven layer, a braided layer, etc.) 1537 that extends towards the distal end of the rigidizing device. FIGS. 15C-15D illustrate the example of a coil-wound tube 1500′ in which the variable stiffness layer is not shown, exposing the coil wound layer, which forms a tube of polymeric material within which the wound coil extends. In general, the one or more coils of the coil wound tubes may have a varying pitch in the non-pressurized distal end regions of the devices. This, coupled with the reduction in wire count and the elimination of rigidization elements in that zone, may reduce bending stiffness of the distal end regions.

Any of the rigidizing apparatuses, including the rigidizing devices/rigidizing members described herein may include an inner coil wound tube and/or an outer coil wound tube configured as a variable pitch coil wound tube. This feature may provide a tip that transitions between devices having different stiffnesses (e.g., between rigidized and rigidized configurations) without damaging the devices and without snagging or catching.

For example, described herein are rigidizing apparatuses including an inner coil wound tube, an outer coil wound tube, a variable stiffness layer and a bladder layer. The bladder layer is configured to engage the variable stiffness layer to alter the stiffness of the device based on the applied pressure (e.g., positive pressure or in some examples negative pressure). For example, the bladder layer may be configured to be pressurized by positive or negative pressure to drive the bladder layer against the variable stiffness layer. In general, the inner coil in either or both the inner coil wound layer and the outer coil wound layer may be configured so, over a distal end region of the rigidizing device that is not rigidizing (e.g., the distal most 0.5 cm, 1 cm, 2 cm, 3 cm, 4 cm, 5 cm, 6 cm, etc. or more) the coil extends as a single member that extends helically around the distal end region with a pitch that increases from the pitch over the rigidizing region. In some examples the pitch may increase continuously. In some examples the apparatus includes just an inner coil wound member (ICW) having two layers of coils; the distal end region either the top coil or the bottom coil may extend distally with an increased or increasing pitch, while the opposite coil (e.g., the bottom coil or the top coil) stop proximal of the distal end region.

### Reflowed Distal Tips

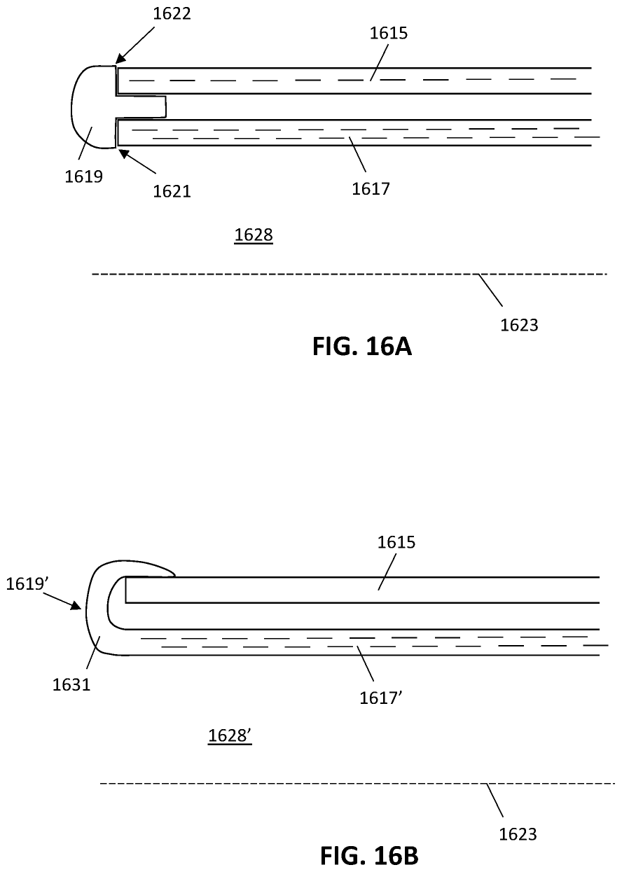

Any of the rigidizing devices (or rigidizing members, which may be part of a rigidizing apparatus) may include a tip configured as a cap or seal around and/or between the layers forming the rigidizing device. FIG. 16A illustrates an example of a section through a portion of a distal end portion of a rigidizing device that includes an inner coil wound tube 1617 (ICWT), which may be any of the coil wound tubes described herein, and an outer coil wound tube 1615 (OCWT). In this example the other layers (e.g., bladder layer, variable stiffness layer, etc.) are not shown but may be included. In FIG. 16 the section shows a cross-sectional view through just the upper portion of the tubular rigidizing device as indicated by the axial midline 1623. In FIG. 16A an annular tip 1619 (e.g., cap, ring, etc.) is attached over the distal ends of both the ICWT and the OCWT, extending between them, and covering and/or sealing the distal end, preventing access into the layered region. This ring-shaped tip 1619 may extend over the inner and outer coil-wound tubes, which in some examples, may result in a lip or edge 1621 on the inner and/or outer surface. In FIG. 16A the lip 1621 may extend into the inner lumen 1628. Thus, an object, including but not limited to a second (e.g., inner) rigidizing device that is inserted into the lumen 1628 of the example rigidizing device shown would potentially scrape against this lip or ridge, which may damage the object or may prevent it from moving axially and/or radially as smoothly and easily. Similarly, if this device is being inserted into a larger apparatus a lip or ridge on the outer edge 1622 may also be undesirable for similar reasons.

Thus, it may be preferred to have tip or cap regions that smoothly transition over this distal end region and seal the layers, or seal between the inner and outer most layers, such as an ICWT and an OCWT.

The coil wound layers including the OCWT and ICWT may include a polymeric material surrounding the coil, as described above. In some examples this polymeric material in which the coils are surrounded or embedded may be enhanced to prevent breakage and/or puncture of the coil. In any of these apparatuses the polymeric material surrounding the coils may be reflowed distally to form the annular seal or tip of the rigidizing device. For example,

FIG. 16B illustrates an example of a distal end portion of a rigidizing device similar to that shown in FIG. 16A but with the polymeric material forming part of the inner coil wound tube 1617′ reflowed distally 1631 so that it extends over and is rolled-back onto or over the outer coil wound tube 1615, forming a distal tip (e.g., a reflow tip) 1619′. In this example the inner coil wound tube is reflowed distally and over the distal edge; alternatively or additionally, in some examples the outer coil wound tube 1615 may instead be reflowed distally to form the tip 1619′. The configuration shown in FIG. 16B may be particularly helpful when another device moves axially into or out of the lumen 1628′ of the rigidizing device, e.g., as when the rigidizing device is an outer rigidizing member of a nested robotic apparatus as described above, and may provide material continuity and negates the need to bond in a separate distal end.

In the example shown in FIG. 16B, the reflowed tip may be formed by reflowing the polymeric material of the inner coil wound tube distally to form the tip 1619′, or a portion of the tip, of the rigidizing device. Although FIG. 16B shows an example of a device in which the reflowed tip is inverted and rolls back over the distal end and contacts the outer coil wound tube (and may be sealed, glued or otherwise attached thereto) in some examples the reflowed tip is not inverted or rolled over, but may extend distally forward; in some examples the reflowed tip has a funnel shape.

Thus, in general the distal tip may be formed with a reflowed polymer material. The resulting reflowed tip may reduce or eliminate any discontinuity (e.g., edge, lip, etc.) between the inner and/or outer coil wound tube and the tip of the device. Any appropriate fabrication technique may be used to form the distal tip, including the distal reflowed tip. Reflowing of the polymeric material may refer to thermal reflow that reshapes the polymeric material from an initial shape into the tip profile. For example, the tip may be formed by controlling the tip shape using a mandrel. The mandrel may control the shape, thickness (which may be constant and/or may vary along the length of the tip or distal end region). The material may be chosen to have an appropriate durometer both for biological suitability but also to prevent damage or punction of the tip from device.

FIG. 16C illustrates one example of a section through a portion of a mandrel 1635 that may be used for reflowing the polymeric material of the device to form the reflowed distal tip. In FIG. 16C the inner coil wound tube (ICWT) 1617″ is shown on the mandrel so that the distal end region of the ICWT is flared out on the mandrel to from the polymer tip region 1639. This polymer tip region may be a pre-inversion polymer tip (or funnel-shaped polymer tip). For example, FIG. 16D illustrates an example of a rigidizing device including a funnel-shaped reflowed polymeric tip 1619″ that extends distally. The device includes an outer coil wound tube 1615 and an inner coil would tube (not visible). FIG., 16E illustrates another example of a rigidizing device including a rolled-back reflowed polymeric tip 1619′ that extends over the distal end as an annular ring and is also sealed to the outer catheter wound tube 1615.

### Protected Wire

Any of the rigidizing devices described herein may be configured to reduce or prevent the risk of a wire (e.g., the wire of the coil in a coil wound tube) from breaking and/or protruding from the device, which may risk damaging the patient. For example, any of these apparatuses may include reinforcement at the distal end region where the coil terminates.

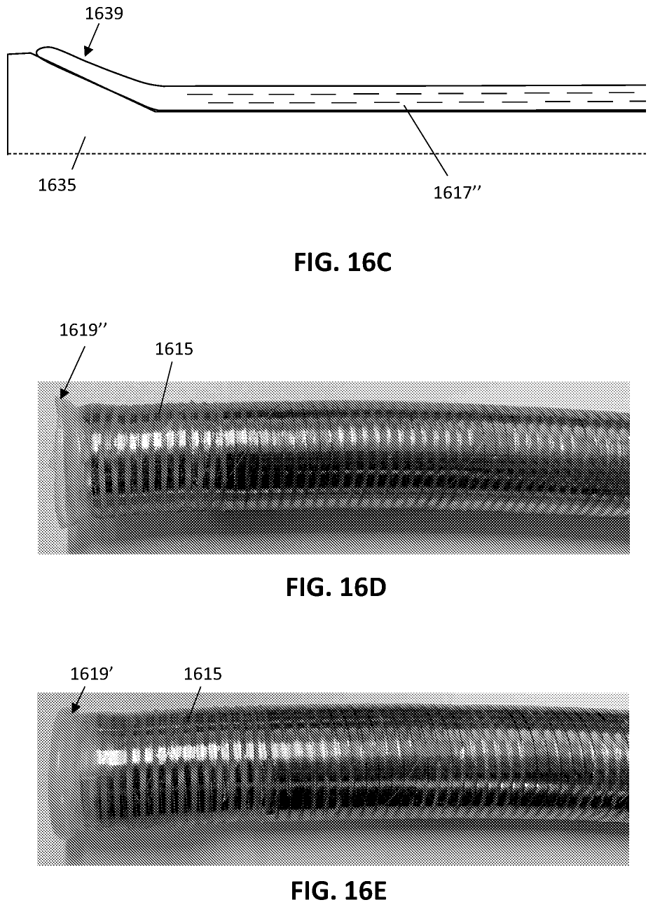

For example, FIG. 17 schematically illustrates one example of a distal end of a rigidizing device 1700 including an outer tube reinforcement layer 1741. In FIG. 17, the apparatus includes a wall volume including an outer reinforced tube 1715 (e.g., an outer coil-reinforced tube), an inner reinforced tube 1717 (e.g., an inner coil-reinforced tube), a bladder layer 1747 and a variable stiffness layer 1745 (e.g., braid layer). The distal end of the device is shown, including the distal tip region 1752. The coil 1716 (e.g., helically wound wire or wires) of the outer reinforced tube are shown, and the coil ends 1716′ proximal to the distal tip of the device. This distal end region of the coil is covered by the reinforcement layer 1741. Any appropriate reinforcement layer may be used, including a polymeric material, metals, etc. The reinforcement layer may be puncture resistant, and relatively thin (e.g., between about 0.0002″-0.002″). Examples of materials that may be used for the reinforcement layer may include: PET, polyimide, PEEK, aluminum, stainless steel, platinum, and gold. In some examples the reinforcement material is a polyimide film. The reinforcement material may sit flush with the outer surface of the device. In some examples the reinforcement material may be secured by an adhesive. Alternatively, in some examples the reinforcement material may be a heat shrink material or a metal band (e.g., stainless steel, platinum, gold, etc.) that may be attached over region including the end of the coil. The reinforcement layer may be any appropriate size (e.g., diameter).

The distal end of any of these apparatuses may be tapered. For example, in FIG. 17 the distal end region may be tapered 1743, which may allow it to be more readily inserted and/or removed from the body. The distal end region may be tapered in order to make space for the addition of the outer tube reinforcement layer, so that the overall outer diameter does not increase in this region or substantially increase the wall thickness and thus increase the outer diameter. The outer diameter may be maintained as substantially the same or may be gradually changed (e.g., tapered) to prevent the formation of a lip or ledge that could cause injury to the patient or prevent fitting through mating devices. The rigidizable region may include a radial gap (e.g., between the bladder layer and outer tube in FIG. 17) to allow the variable stiffness layer to freely move when not rigidized; this may allow the wall thickness to be thinner, e.g. to neck down, in this distal end region. In some examples, as shown in FIG. 17, the distal end region may be distal to the end of the bladder and variable stiffening region, and therefore may not be a rigidizable zone.

In any of these apparatuses, the distal end region, including the distal tip, may be configured so that it does not rigidize, as described above. In addition, any of these apparatuses may be configured so that the distal end is substantially soft. In any of these examples the device may include a longitudinal gap region 1751 between the distal end of the rigidization region and the distal end region of the device. In some examples, the device does not include a longitudinal gap. A longitudinal gap 1751 may increase the flexibility of the distal end region. This may enhance trackabililty.

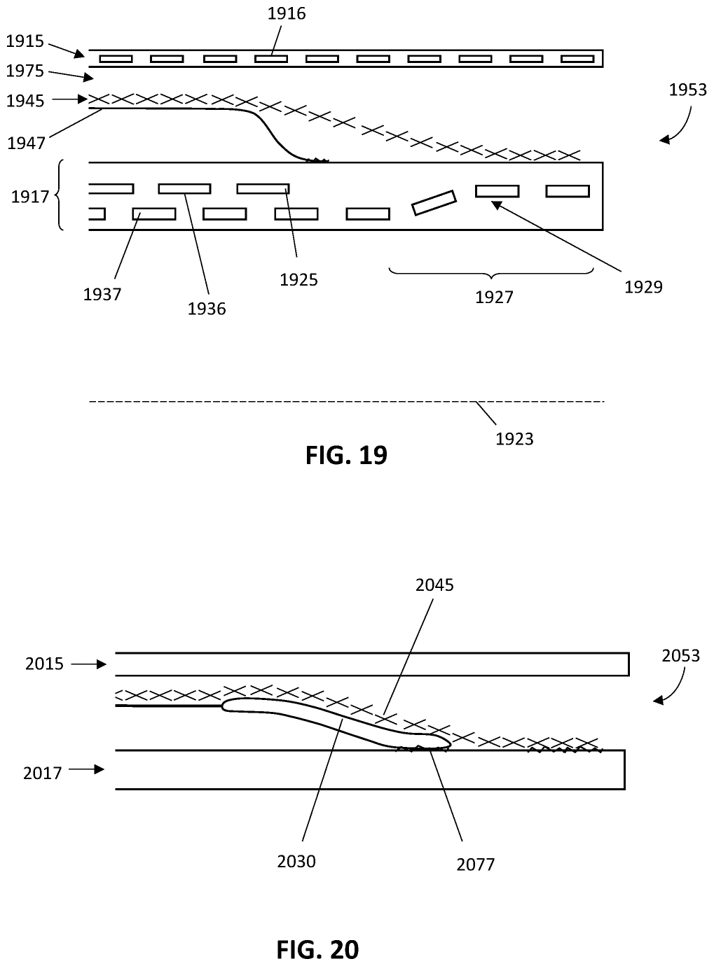

As described above, these apparatuses may be configured to prevent rupture of the reinforcement coils by adding a reinforcement layer. However, in some cases it may not be desirable to add additional material, and potentially additional thickness to this region. For example, in some variations it may be desirable to re-center or adjust the radial position of the coil within the coil wound layer to increase the thickness of surrounding material, e.g. polymeric material, without increasing the overall radial thickness. In any of these examples this may mean that the radial position of the coil of the coil-wound layer (e.g., the inner coil wound layer and/or outer coil wound layer) may be changed (increased or decreased) relative to the more proximal position, so that the end region is centered relative to the surround polymeric material (e.g. the matrix material). This may be particularly useful where the coil-wound layer includes both an inner reinforcing member (e.g., inner coil) and an outer reinforcing member (e.g., outer coil), as described above. In some examples one of the inner or outer reinforcing members (e.g., coils) may terminate more proximally than the other outer or inner reinforcing member (e.g., coil) and the other reinforcing member may be radially centered within the polymeric matrix. This is illustrated in FIG. 19.

FIG. 19 shows an example of a distal end of a portion of a rigidizing device including an outer coil wound tube 1915 and an inner coil wound tube 1917, a variable stiffness layer (e.g., braided layer, woven layer, etc.) 1945 and a bladder layer 1947. In this example the variable stiffness layer extends all the way to the distal end (distally of the bladder layer) and may be attached to the inner coil wound layer. The bladder layer may be sealed against the inner coil wound layer proximal to the distal end. The outer coil wound layer includes a single coil 1916 helically wrapped around the outer coil wound layer and surrounded by a polymeric matrix material. The inner coil wound layer in this example includes a top (e.g. outer) wire 1936 or coil and a bottom (e.g., inner) wire 1937 or coil that are counter-wound along the length of the outer coil-wound tube. In this example the top wire ends proximal to the distal end region 1953 and the bottom wire continues to helically extend distally to the distal end region. In the proximal region having the inner and outer coils the pair of coils may be collectively centered within the polymeric matrix material, as shown in FIG. 19. Once the inner coil wound tube transitions to a single helically wound coil near the distal end region 1927, the radial path of the coil wound tube may be adjusted to center the bottom wire within the polymeric matrix material, as shown, by re-directing the wire 1929 at the distal end. In some examples the pitch of the re-directed wire may be the same. In some examples, as described in FIGS. 15A-15D above, the pitch may change.

In FIG. 19, the overall thickness of the inner coil wound tube remains constant, even at the distal end. In some cases the thickness may be increased or decreased in some cases, in order to keep the thickness of the inner coil wound tube relatively constant (e.g., ±5% or less, ±7% or less, ±10% or less, etc.) polymeric material may be added to this region. The transition from the pair of wound coils in the inner coil wound layer may also enhance flexibility of the distal end region of the rigidizing device, which may be desirable, as described above, as helping normalize the transition between rigid and non-rigid devices moving relative to each other (e.g. nested devices).

In any of the rigidizing devices described herein the wall thickness of the device may include a gap or region within which the variable stiffness layer may move when not compressed by the bladder layer. For example in FIG. 19, the gap region 1975 is shown between the outer coil-wound tube 1915 and the variable stiffness layer 1945; pressurizing the bladder layer to rigidize the device causes the bladder layer to drive the variable stiffness layer against the outer coil wound tube 1915 and reduces the volume of the gap region 1975. Alternatively, in some examples the device is configured so that pressurizing the bladder layer 1947 (which may be attached to the outer coil wound tube or another radially outward layer) causes the bladder layer to drive the variable stiffness layer against the inner coil wound tube across the gap layer. This gap layer may be an air gap or may be filled with any other fluid (e.g., water, etc.). The gap layer may be coupled to one or more vents, e.g., proximal vents, to allow release of fluid (e.g., air) as the bladder layer is pressurized.

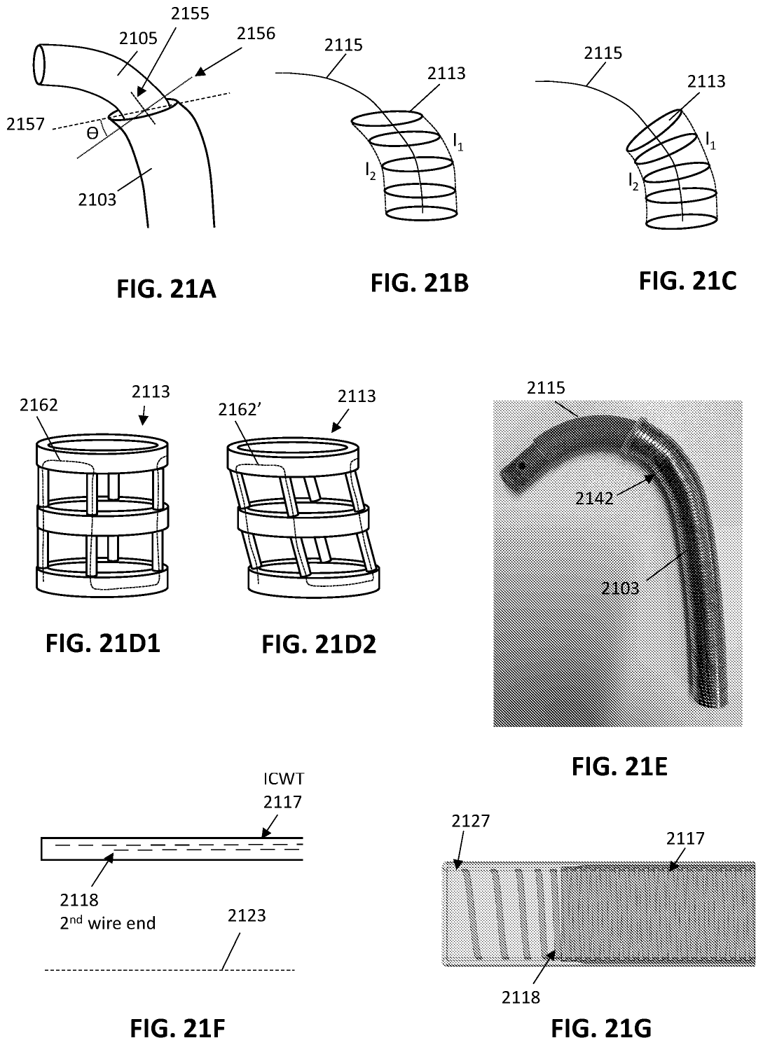

In some examples, one or more other layers may be increased or expanded in order to prevent breakage and/or rupture of components of the rigidizing device. In some examples the bladder layer may be reinforced or thickened, e.g., by doubling over itself one or more times, by adding one additional material, etc. In some examples additional reinforcing material may be added, such as a different material having a higher durometer. For example, FIG. 20 illustrates a portion of a distal end section through a rigidizing device including an inner coil wound tube (ICWT) 2017, an outer coil wound tube (OCWT) 2015, a variable stiffness layer 2045 and a bladder layer 2030. In this example the bladder layer is shown enlarged over the distal end region. In FIG. 20 the variable stiffness layer extends distally of the end of the bladder layer and is attached to the ICWT. The reinforced distal end of the bladder layer may be secured (sealed, glued, bonded, etc.) to the ICWT proximal to the distal end 2053 of the device. Such reinforcements can be created at either end, where these additive elements are created separate from the bladder as it exists in the mid-zone region.

In rigidizing devices such as those shown in FIG. 20, the thicker bladder on distal/proximal region may enhance the strength of the distal end region and may be formed by doubling the bladder layer back over itself. For example, the bladder may be extruded in in a single length and bonded onto an ICWT. Reinforcing the bladder layer may prevent rupture of the seal and/or the bladder at the distal end region under pressure, as the transition zone between the bond and the bladder may otherwise be less robust, while experiencing the highest amount of stress when the bladder is pressurized. In some cases the bladder layer distal end region may be reinforced by everting the bladder layer at the distal end and re-melting the material forming the bladder layer. Alternatively, additional material (including the same material forming the bladder layer or a different material) may be added.

In the example shown in FIG. 20 the bladder may be bonded 2077 (e.g., thermally bonded and/or adhesively bonded) to the ICWT. Bonding this may create a high strain region that may be improved by reinforcing as described above. The reinforced region may be any appropriate length. For example, the reinforced region may be between about 0.25 fold, 0.5 fold and 5 fold of the diameter of the ICWT. In some examples the reinforced region is about 0.4 inches (0.5 inches, 0.6 inches, 0.7 inches, etc.).

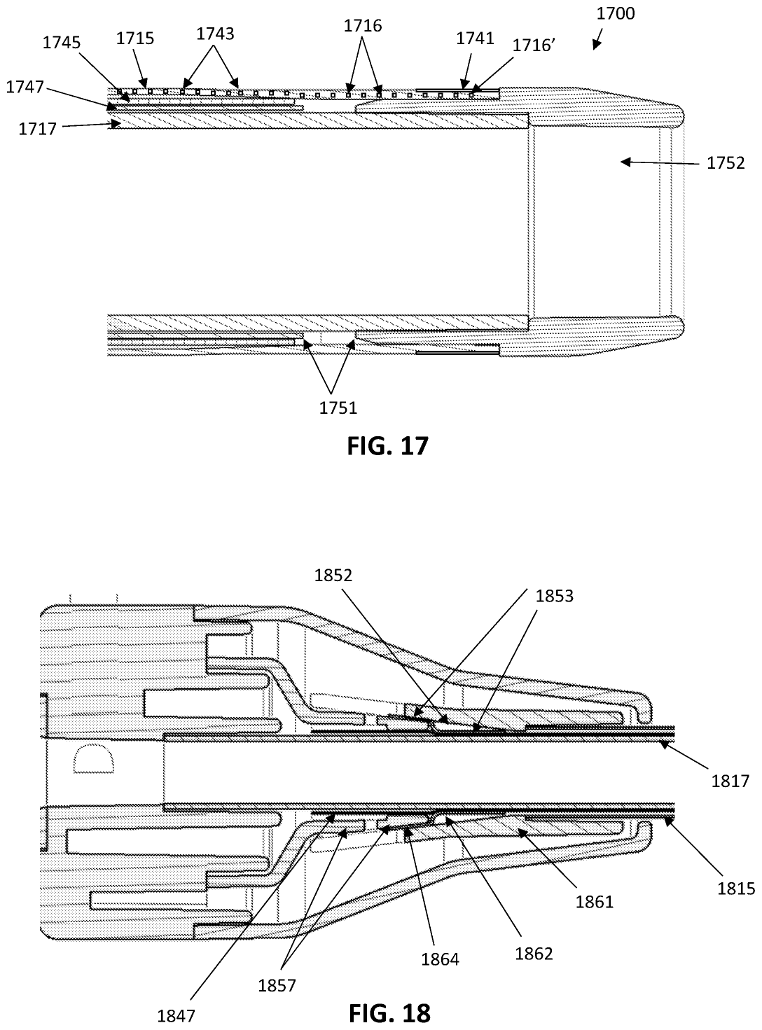

Any of the apparatuses (e.g., including any of the rigidizing devices) described herein may include a proximal end that is configured to allow the application of pressure (positive and/or negative pressure) to rigidize the device as described herein, while preventing damage or failure of the device. For example, FIG. 18 illustrates one example of a proximal end region of a device showing the interface for the inner tube 1817, the outer tube 1815, and the bladder region 1847. The variable stiffness layer (not shown in FIG. 18) may be attached from the proximal end region or from a more distal position.

In FIG. 18, the outer tube 1815 is coupled to an outer tube adapter 1861 that fits the bladder 1847 that is coupled at the proximal end to a bladder adapter 1857. The bladder adapter may be configured to provide passage on one size for the application of pressure (e.g. positive pressure) while preventing rupture or disruption of the bladder. In some cases the proximal end region of the bladder may be a relatively weaker region that may be ruptured when pressure is applied. In some examples the bladder attachment to the proximal end of the device may be reinforced, by a bladder reinforcement film 1853. This bladder reinforcement film may be bonded 1864 over the proximal end of the bladder material and the bladder adapter, as shown in FIG. 18. In particular, the bladder reinforcement film may prevent rupture over gap regions 1852 which may occur where the bladder, bladder adapter and/or outer tube adapter meet. The bladder reinforcement film may be any appropriate material, including additional (or thicker) bladder material (e.g., polymeric material), materials having a higher durometer than the bladder material, or materials having a lower durometer than the bladder material.

### Parallelogram Tip Configurations

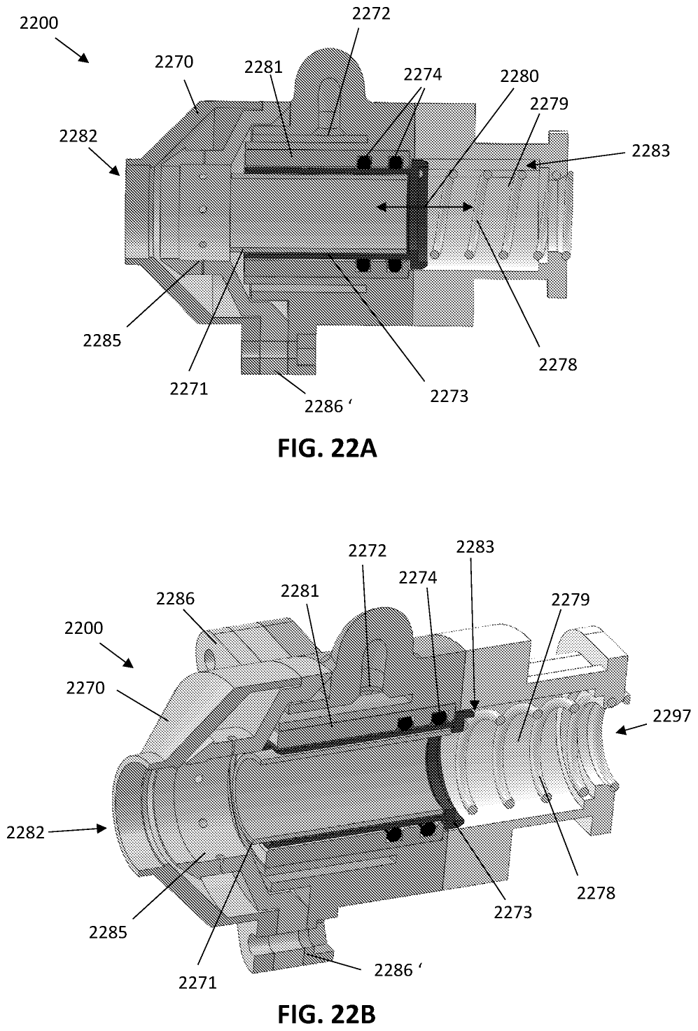

The rigidizing devices described herein may be configured so that they bend in the un-rigidized configuration at the distal end region without compressing significantly compressing or reducing the length of the distal end. As a result, this may allow the distal end region to bend by laterally displacing the distal opening (“parallelogramming”), as illustrated in FIG. 21. Parallelogramming refers to bending in which the radially opposite sides of the rigidizing device (“tube”) may remain substantially parallel. In FIG. 21 a rigidizing apparatus includes an inner rigidizing member 2105 that is shown telescoping out of an outer rigidizing member 2103. The outer rigidizing member may be configured to bend at the distal end region by parallelogramming, so the lengths of the radially opposite sides of the outer rigidizing member remain substantially the same length as the outer rigidizing member is bent. As a result, the bending may occur in a sliding movement in which the plane of the distal opening does not remain parallel to the long axis of the inner rigidizing member as the inner rigidizing member exits the lumen of the outer member. As shown in FIG. 21A, a line 2156 perpendicular to the long axis 2155 of the inner rigidizing member as it leaves the outer rigidizing member forms an angle (O) with respect to the plane of the distal opening 2157 of the outer rigidizing member. This angle (e) will increase as the bend increases (up to a limit) in a parallelogramming configuration, whereas in a device that compresses the sides. This is illustrated schematically in FIGS. 21B and 21C. FIG. 21B schematically illustrates an example of a parallelogramming apparatus in which the lengths 11, 12 of the radially opposite sides of the distal end region of the outer rigidizing device remain approximately equivalent (e.g., within ±5% or less, 7% or less, etc.) as the distal end region is bent. In this example the plane of the distal opening 2113 of the schematic device is not perpendicular to the long axis of the inner member as it extends distally out of the outer rigidizing device. A line 2115 illustrates the long axis of the inner member (e.g., inner catheter device) as it leaves the outer device 2113. In contrast FIG. 21C shows an example of an apparatus that bends by compressing a first side that is radially opposite a second side (and/or expanding the radially opposite side, so that the lengths of the sides (11, 12) become increasingly different as bending of the apparatus occurs.

FIGS. 21D1 and 21D2 illustrate the operation of parallelogramming, showing the resulting radial sliding movement that occurs rather than compression of a proton of the sides of the example shown. In FIG. 21D1 an example of a distal end region that configured to bend by parallelogramming, while FIG. 21D2 shows a distal end region that is configured to bend as expected, by bending without substantially altering the length of the sides 2162. In FIG. 21D1 the length 2162 of a path between the proximal and distal end is approximately the same as the length 2162′ in the bent configuration.

FIG. 21E illustrates an example of a rigidizing device 2103 that is bent, in a non-rigidized configuration, over the distal end of a tool 2115. In this example a portion 2142 of the outer coil-wound tube bends by parallelogramming.

Parallelogramming may be beneficial to the operation of a rigidizing member. Thus, any of these rigidizing devices and methods of using them may be configured to bend by parallelogramming. In some instances, the additional bending stiffness results in parallelogramming. For example, any of the apparatuses described herein may be configured to result in parallelogramming when bending the distal end region. For example, the apparatus may be configured so that the variable stiffness layer (e.g., brain, weave, etc.) may extend distal to the bladder layer; in some examples, the variable stiffness layer may extend to the distal end of the apparatus. The variable stiffness layer may limit the ability of the distal end of the device to compress or extend in this local region, which may result in parallelogramming. In any of these examples the distal end region of the apparatus may also or alternatively be configured so that if there are dual coils (e.g., an upper coil and a lower coil) in the outer and/or inner coil reinforced member, either the upper or the lower coil may end prior to the distal end region, as described above, and shown in FIG. 21F. In this example, the inner coil wound tube 2117 includes a pair of counter wound coils and the inner coil 2118 ends prior to the distal end (for reference the midline 2123 is shown). FIG. 21G also illustrates an example in which the distal end region is made further flexible by increasing the pitch of the coil that extends distally in the distal end region after the second, counter wound coil terminates 2118; in this example the variable stiffness layer 2127 (e.g., woven layer, knitted layer, braided layer, etc.) extends to the distal end of the device.

### Axial Accommodation

Any of the apparatuses described herein may include an axial accommodator to account for and release any stain resulting from rigidizing the apparatus. In general, applying pressure (positive or in some examples, negative, pressure) may result in axial expansion of the length of the device. This could relate to length changes as braid is acted up by a pressurized bladder. Although this axial expansion may be small (e.g., 3 mm or less, 2 mm or less, 1 mm or less) the resulting strain may risk damage to the apparatus, particularly at the distal or proximal ends of the apparatus. This problem may be particularly acute when the apparatus includes a torsional stiffening member extending the length of the device and providing torsional (e.g., rotational) stiffness.

Thus, any of these apparatuses may include an axial accommodator to accommodate for axial expansion and contraction (e.g., when rigidizing/de-rigidizing) the rigidizing device. In any of these examples the axial accommodator may be configured to couple to the proximal end of the rigidizing device, and may couple to the one or more layers forming the rigidizing device. In particular the apparatus may differentially couple to a subset of the one or more layers to allow relatively axial movement between some of the layers. The axial accommodator may include a housing and/or one or more internal couplers to couple to the internal layers (e.g., the outer coil wound tube and the inner coil would tube), and a biased expansion element that may be configured to deflect axially to prevent or release strain that may be coupled to an internal layer, such as a torsional stiffening layer. The axial accommodator may include a housing that may also receive and couple to a source of pressure (to deliver positive and/or negative pressure to the bladder layer to rigidizing the apparatus). The axial accommodator may include a sled or shuttle (e.g. a torsional accommodator shuttle) that may move relative to the housing and may couple (e.g., rigidly couple) to an internal layer such as a torsional stiffening layer, within the rigidizing device while the inner and/or outer region of the device is coupled to the housing and/or to a connector. The device may include a bias (e.g., spring) to restore the position of the shuttle or sled, and therefore the position of the internal layer after the rigidizing layer is transitioned to the flexible state.

Thus an axial accommodator may be configured to allow a natural change in the length of the rigidizing device.

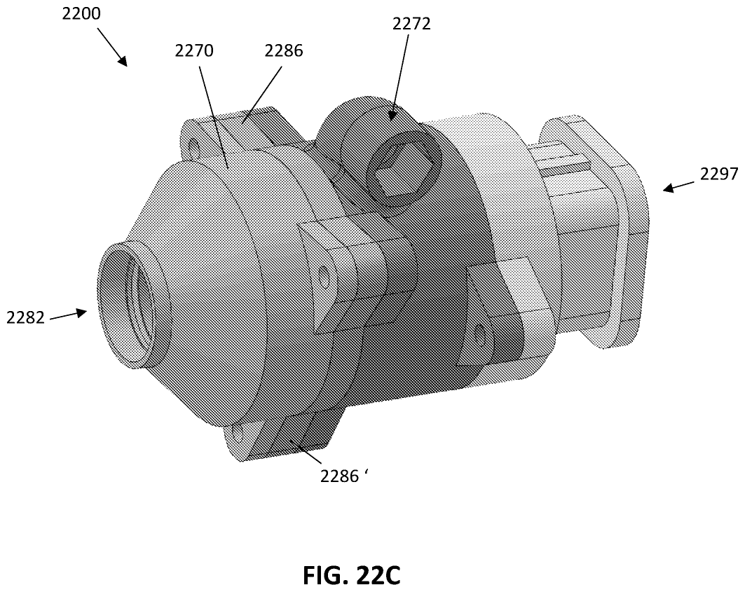

FIGS. 22A-22C illustrate one example of an axial accommodator. In this example, the axial accommodator 2200 includes a housing 2270 (e.g., an axial accommodator housing) that includes a distal opening 2282 into which the proximal end of the apparatus may be inserted. Within the housing a connector 2285 is configured to couple to the outer and/or an inner layer of the rigidizing device. The axial accommodator in this example also includes a shuttle 2273 (e.g., an axial expansion/accommodator shuttle) that may rigidly couple to an internal layer of the rigidizing device, such as a torsional stiffening layer 2271 (e.g., a torsionally stiff internal structure, a portion of which is shown for reference in FIGS. 22A-22B). The shuttle 2273 may be movably held within a collet 2281 so that the shuttle may move in an axial direction (in/out) in the long axis of the device 2280. The axial accommodator also includes an expansion chamber 2279 into which the shuttle may slide. The expansion chamber (which may be formed by the housing) may also include a rail or track allowing axial movement, but preventing rotation or other degrees of freedom. The axial accommodator also includes a return bias 2278 (e.g., in this example a coil spring, though other return biases may be used).

In any of these apparatuses the positive or negative pressure to rigidize the device may be applied through the axial accommodator. For example, the axial accommodator may include a pressure inlet 2272 into which pressure may be applied and directed, via one or more channels within the axial accommodator housing body, to the region of the rigidizing device including the bladder, so that the bladder may be driven against the variable stiffness layer, to rigidize the device. The axial accommodator may also include one or more seals (e.g. o-rings) between the shuttle 2273 and the collet 2281 to prevent loss of pressure.

In general, these axial accommodators may also be configured as annular devices that may provide an opening through the device to allow passage into and out of the lumen of the rigidizing device.

FIG. 22C shows an external view of the axial accommodator of FIGS. 22A-22B, showing the central lumen 2297 and the pressure port 2272, as well as a pair of flanges 2286, 2286′ formed on/in the housing. The flanges may allow gripping or attachment of the axial accommodator for manual or automatic movement (e.g., rotational movement, axial movement, etc.) of the device.

As mentioned above the axial accommodator may couple to the proximal end of the device and may allow for changes in the length of one or more layers (e.g., the torsional stiffening member length) during operation of the device, e.g., rigidizing/de-rigidizing the rigidizing device. In some example the axial accommodator may be permanently coupled to the proximal end of the device; in other examples the axial accommodator may be releasably or removably attached. In use, when pressure (e.g., positive and/or negative pressure) is applied to the rigidizing device, e.g., through the pressure inlet, to deflect the bladder layer and apply force against the variable stiffness layer, which may in turn cause axial expansion of the torsional stiffening layer, the torsional stiffening layer coupled to the shuttle may be deflected axially (e.g., proximally) relative to the housing of the axial accommodator by sliding (constrained to the axial movement without rotation) into the expansion chamber, pushing against the return bias. Once the rigidizing device is de-rigidized, e.g., by reducing or removing the pressure in the inlet, the return bias may drive the shuttle, coupled to the torsional stiffening layer distally again. In this manner, the axial accommodator may allow lateral (axial) accommodation of movement of the torsional stiffening layer, thereby relieving strain on the apparatus.

It should be understood that any feature described herein with respect to one embodiment can be combined with or substituted for any feature described herein with respect to another embodiment. For example, the various layers and/or features of the rigidizing devices described herein can be combined, substituted, and/or rearranged relative to other layers.

It should be appreciated that all combinations of the foregoing concepts and additional concepts discussed in greater detail below (provided such concepts are not mutually inconsistent) are contemplated as being part of the inventive subject matter disclosed herein and may be used to achieve the benefits described herein.

The process parameters and sequence of steps described and/or illustrated herein are given by way of example only and can be varied as desired. For example, while the steps illustrated and/or described herein may be shown or discussed in a particular order, these steps do not necessarily need to be performed in the order illustrated or discussed. The various example methods described and/or illustrated herein may also omit one or more of the steps described or illustrated herein or include additional steps in addition to those disclosed.

When a feature or element is herein referred to as being “on” another feature or element, it can be directly on the other feature or element or intervening features and/or elements may also be present. In contrast, when a feature or element is referred to as being “directly on” another feature or element, there are no intervening features or elements present. It will also be understood that, when a feature or element is referred to as being “connected”, “attached” or “coupled” to another feature or element, it can be directly connected, attached or coupled to the other feature or element or intervening features or elements may be present. In contrast, when a feature or element is referred to as being “directly connected”, “directly attached” or “directly coupled” to another feature or element, there are no intervening features or elements present. Although described or shown with respect to one embodiment, the features and elements so described or shown can apply to other embodiments. It will also be appreciated by those of skill in the art that references to a structure or feature that is disposed “adjacent” another feature may have portions that overlap or underlie the adjacent feature.

Terminology used herein is for the purpose of describing particular embodiments only and is not intended to be limiting of the invention. For example, as used herein, the singular forms “a”, “an” and “the” are intended to include the plural forms as well, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof. As used herein, the term “and/or” includes any and all combinations of one or more of the associated listed items and may be abbreviated as “/”.

Spatially relative terms, such as “under”, “below”, “lower”, “over”, “upper” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if a device in the figures is inverted, elements described as “under” or “beneath” other elements or features would then be oriented “over” the other elements or features. Thus, the exemplary term “under” can encompass both an orientation of over and under. The device may be otherwise oriented (rotated 90 degrees or at other orientations) and the spatially relative descriptors used herein interpreted accordingly. Similarly, the terms “upwardly”, “downwardly”, “vertical”, “horizontal” and the like are used herein for the purpose of explanation only unless specifically indicated otherwise.

Although the terms “first” and “second” may be used herein to describe various features/elements (including steps), these features/elements should not be limited by these terms, unless the context indicates otherwise. These terms may be used to distinguish one feature/element from another feature/element. Thus, a first feature/element discussed below could be termed a second feature/element, and similarly, a second feature/element discussed below could be termed a first feature/element without departing from the teachings of the present invention.

Throughout this specification and the claims which follow, unless the context requires otherwise, the word “comprise”, and variations such as “comprises” and “comprising” means various components can be co-jointly employed in the methods and articles (e.g., compositions and apparatuses including device and methods). For example, the term “comprising” will be understood to imply the inclusion of any stated elements or steps but not the exclusion of any other elements or steps.

In general, any of the apparatuses and methods described herein should be understood to be inclusive, but all or a sub-set of the components and/or steps may alternatively be exclusive and may be expressed as “consisting of” or alternatively “consisting essentially of” the various components, steps, sub-components or sub-steps.

As used herein in the specification and claims, including as used in the examples and unless otherwise expressly specified, all numbers may be read as if prefaced by the word “about” or “approximately,” even if the term does not expressly appear. The phrase “about” or “approximately” may be used when describing magnitude and/or position to indicate that the value and/or position described is within a reasonable expected range of values and/or positions.

For example, a numeric value may have a value that is ±0.1% of the stated value (or range of values), ±1% of the stated value (or range of values), ±2% of the stated value (or range of values), ±5% of the stated value (or range of values), ±10% of the stated value (or range of values), etc. Any numerical values given herein should also be understood to include about or approximately that value, unless the context indicates otherwise. For example, if the value “10” is disclosed, then “about 10” is also disclosed. Any numerical range recited herein is intended to include all sub-ranges subsumed therein. It is also understood that when a value is disclosed that “less than or equal to” the value, “greater than or equal to the value” and possible ranges between values are also disclosed, as appropriately understood by the skilled artisan. For example, if the value “X” is disclosed the “less than or equal to X” as well as “greater than or equal to X” (e.g., where X is a numerical value) is also disclosed. It is also understood that the throughout the application, data is provided in a number of different formats, and that this data, represents endpoints and starting points, and ranges for any combination of the data points. For example, if a particular data point “10” and a particular data point “15” are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

Although various illustrative embodiments are described above, any of a number of changes may be made to various embodiments without departing from the scope of the invention as described by the claims. For example, the order in which various described method steps are performed may often be changed in alternative embodiments, and in other alternative embodiments one or more method steps may be skipped altogether. Optional features of various device and system embodiments may be included in some embodiments and not in others. Therefore, the foregoing description is provided primarily for exemplary purposes and should not be interpreted to limit the scope of the invention as it is set forth in the claims.

The examples and illustrations included herein show, by way of illustration and not of limitation, specific embodiments in which the subject matter may be practiced. As mentioned, other embodiments may be utilized and derived there from, such that structural and logical substitutions and changes may be made without departing from the scope of this disclosure. Such embodiments of the inventive subject matter may be referred to herein individually or collectively by the term “invention” merely for convenience and without intending to voluntarily limit the scope of this application to any single invention or inventive concept, if more than one is, in fact, disclosed. Thus, although specific embodiments have been illustrated and described herein, any arrangement calculated to achieve the same purpose may be substituted for the specific embodiments shown. This disclosure is intended to cover any and all adaptations or variations of various embodiments. Combinations of the above embodiments, and other embodiments not specifically described herein, will be apparent to those of skill in the art upon reviewing the above description.

1.-76. (canceled)

77. A rigidizing apparatus, the device comprising:
an elongate rigidizing body configured convert between a flexible configuration into a more rigid configuration by applying pressure; and
an axial accommodator comprising:
a body coupled to a first portion of a proximal end of the elongate rigidizing body;
a shuttle coupled to a second portion of the proximal end of the elongate rigidizing body, wherein the shuttle is configured to move axially relative to the body to release strain resulting from rigidizing the apparatus; and
a return bias driving the shuttle.

78. The apparatus of claim 77, wherein the axial accommodator further comprises a pressure inlet in fluid communication at least the bladder layer.

79. The apparatus of claim 77, wherein the axial accommodator further comprising a collet configured to slidably engage with the shuttle.

80. The apparatus of claim 79, wherein the axial accommodator further comprising one or more sealing members between the shuttle and the collet.

81. The apparatus of claim 77, wherein the axial accommodator further comprises an expansion chamber within the body configured to receive the shuttle.

82. The apparatus of claim 77, wherein the axial accommodator further comprises one or more rails within the body to prevent rotational movement of the shuttle.

83. The apparatus of claim 77, wherein the axial accommodator further comprising a central lumen extending through the axial accommodator in fluid communication with a lumen of the elongate rigidizing body.

84. The apparatus of claim 77, wherein the shuttle is coupled to a torsional stiffening member of the elongate rigidizing body.

85. The apparatus of claim 84, wherein the torsional stiffening member comprises a torsional stiffening layer.

86. The apparatus of claim 77, wherein the elongate rigidizing body is configured to rigidize by applying positive pressure.

87. The apparatus of claim 77, wherein the elongate rigidizing body is configured to rigidize by applying negative pressure.

88. The apparatus of claim 77, wherein the elongate rigidizing body comprises a plurality of tubular layers including an outer layer, a variable stiffness layer comprising a plurality of strand lengths that cross each other, and a bladder layer.

89. The apparatus of claim 77, wherein the first portion of the elongate rigidizing body comprises an outer layer.

90. The apparatus of claim 77, wherein the axial accommodator is permanently attached to the proximal end of the elongate rigidizing body.

91. The apparatus of claim 77, wherein the axial accommodator is removably coupled to the proximal end of the elongate rigidizing body.

92. A rigidizing apparatus, the device comprising:
an elongate rigidizing body comprising a plurality of tubular layers including an outer layer, a variable stiffness layer comprising a plurality of strand lengths that cross each other, and a bladder layer, wherein the elongate rigidizing body is configured convert between a flexible configuration into a more rigid configuration by applying pressure against the bladder layer; and
an axial accommodator comprising:
a body coupled to a first portion of a proximal end of the elongate rigidizing body;
a shuttle coupled to a second portion of the proximal end of the elongate rigidizing body, wherein the shuttle is configured to move axially relative to the body to release strain resulting from rigidizing the apparatus; and
a return bias driving the shuttle.

93. A rigidizing apparatus, the device comprising:
an elongate rigidizing body configured convert between a flexible configuration into a more rigid configuration by applying pressure, the elongate rigidizing device including a torsional stiffening member; and
an axial accommodator comprising:
a body coupled to a first portion of a proximal end of the elongate rigidizing body;
a shuttle coupled to the torsional stiffening member of the elongate rigidizing body, wherein the shuttle is configured to move axially relative to the body to release strain resulting from rigidizing the apparatus; and
a return bias driving the shuttle.
